(19) United States
(12) Patent Application Publication
(10) Pub. No.: US 2021/0315921 A1
(43) Pub. Date: Oct. 14, 2021

(54) Compounds and Methods for Modifying iAge

(71) Applicant: Edifice Health, Inc,, San Mateo, CA (US)

(72) Inventors: David Furman, San Francisco, CA (US); Kevin Schneider, Petaluma, CA (US)

(73) Assignee: Edifice Health, Inc., San Mateo, CA (US)

(21) Appl. No.: 17/226,778

(22) Filed: Apr. 9, 2021

Related U.S. Application Data
(60) Provisional application No. 63/151,280, filed on Feb. 19, 2021.
(60) Provisional application No. 63/016,197, filed on Apr. 27, 2020.
(60) Provisional application No. 63/009,379, filed on Apr. 13, 2020.

(51) Int. Cl. A61K 31/736; A61K 31/592; A61K 31/455; A61K 33/32; A61K 31/015; A61K 31/047; A61K 33/30; A61K 31/198; A61K 31/404; A61K 33/26; A61K 31/4188; A61K 31/522; A61P 35/00

(52) U.S. Cl. CPC A61K 31/736; A61K 31/592; A61K 31/455; A61K 33/32; A61K 31/015; A61K 31/047; A61P 35/00; A61K 31/198; A61K 31/404; A61K 33/26; A61K 31/4188; A61K 31/522; A61K 33/30

(57) ABSTRACT

The compounds and methods described herein can improve iAge (Inflammatory Age) of patients with a specific immunotype. For example, a patients iAge can be moved into a responders cohort from a non-responders cohort for an immunotherapy. The compounds and methods described herein can also improve cardiovascular patient outcomes using cAge to stratify CVD patients into risk cohorts for therapy and monitoring. Higher risk CVD patients can be converted to lower risk patients by treating the patients with molecules that reduce their cAge.

### BACKGROUND

Over the past five years cancer immunotherapy treatments have witnessed a great deal of clinical success in multiple cancer types often with extended disease-free survival periods of >10 years. Examples of successful immunotherapies are immune checkpoint inhibitors, which have demonstrated unprecedented rates of durable responses in many difficult-to-treat cancers. However, regardless of the organ affected and cancer type, only a limited percentage of patients (˜20%) benefit from these approaches. Thus, there is a growing need to identify biomarkers that will improve the selection of patients who will respond to therapy.

Biomarkers are needed both before and during treatment to enable identification of patients likely to respond to immunotherapy treatment in order to reduce inappropriate drug use. Objective clinical responses are defined as a reduction in tumor size during the course of treatment. Multiple baseline factors associated with disease prognosis have been linked to response rates. For example, patients with small-sized tumors or low baseline levels of serum lactate dehydrogenase (LDH) are more likely to respond to anti-PD-1 treatment. Circulating tumor DNA (ctDNA) that can be released by dead tumor cells and detected in the serum of some patients correlate strongly with tumor progression.

Response to anti-PD-1 treatment can partially be predicted by the expression of the ligand PD-L1 within the tumor microenvironment. Although PD-L1 expression is correlated with treatment efficacy in melanoma patients, it is not in patients with other cancers such as squamous cell carcinoma, non-small cell lung cancer and Merkel cell carcinoma.

The presence of neoantigens on tumor cells promotes immunogenicity against tumors and improves treatment efficacy. Thus, high genetic variation between tumor cells and host cells is one indicator of checkpoint inhibitor treatment efficacy. This is particularly true for anti-CTLA-4 treatment in melanoma patients and anti-PD-1 treatment in patients with colorectal cancer or non-small cell lung cancer with high mutation rates.

Other immunological factors associated with improved treatment responses prior to immunotherapy treatment include elevated eosinophil and lymphocyte counts; high numbers of CD8⁺ T cells infiltrating the tumor, and increased TGF-β levels in the serum from melanoma patients treated with anti-PD-1.

A number of post-treatment immune biomarkers have also been suggested to be associated with improved responses to cancer immunotherapy. For instance, patients who were more likely to respond to anti-CTLA-4 treatment had increased counts of inducible co-stimulatory molecule (ICOS)(+) T cells and lower neutrophil/lymphocyte ratios.

### SUMMARY

The disclosure describes a method for treating cancer patients with immunotherapy whereby subjects can be stratified based on their inflammatory age levels; and can receive individualized interventions to reduce inflammatory age and improve clinical and immune responses to cancer immunotherapy treatments.

An inflammatory age scoring system (iAge) can be used to classify cancer patients into those who will mount an objective clinical response to immunotherapy versus those who will not. The inflammatory age scoring system can be used to guide initial therapy targeting inflammation to enable optimal objective responses in those patients who were classified as non-responders. A cytokine response score (CRS) can be used to classify cancer patients into those who will mount an objective clinical response to immunotherapy versus those who will not.

The disclosure also describes a method for treating cardiovascular disease patients or patients at risk of cardiovascular disease whereby subjects can be stratified based on risk for cardiovascular disease based on their inflammatory factor level; and can receive individualized interventions to treat and/or reduce the inflammatory factors and improve their risk profile, cardiovascular health, and response to cardiovascular treatments.

The disclosure also describes methods of improving health, well being and longevity of patients by reducing the iAge score of certain markers so that the patients iAge score is lowered. Described herein are immunotypes that can be used to stratify patients into groups with similar iAge characteristics. Described herein are ten (10) different immunotypes. Four of the immunotypes subclassify those patients who have a significant decrease in iAge from their chronological age. The other six (6) immunotypes classify the remainder of the patients. Subjects in each immunotype can receive similar interventions to improve iAge of any subject with that immunotype. Thus, patients of the same immunotype can be provided with a treatment based upon their immunotype. Each immunotype treatment reduces the iAge score for patients in that immunotype which improves patient health, well-being and longevity.

An inflammatory age scoring system (iAge) can be used to classify patients into those who have higher risk for cardiovascular disease versus those who have a low risk. The inflammatory age scoring system can be used to guide initial therapy targeting inflammation to improve outcomes of patients receiving treatment for cardiovascular disease, and to reduce risk of cardiovascular disease in asymptomatic patients (e.g., prophylactic treatment). MIG, EOTAXIN, Mip-1α, LEPTIN, IL-1β, IL-5, IFN-α and IL-4 (positive contributors) and TRAIL, IFN-γ, CXCL1, IL-2, TGF-α, PAI-1 and LIF (negative contributors) are related to iAge and can be used to make up the iAge score. MIG, LIF and Sirtuin-3 are strongly related to cardiac aging and risk for cardiovascular disease and can be used alone or combination with other factors to define the risk level of a patient.

The disclosure describes a method for treating subjects with immunotherapy (e.g., cancer patients), vaccines (e.g., subjects who will benefit from vaccination), and antipathogen therapeutics (e.g., antibiotics, antivirals, antifungals, etc.) whereby subjects can be stratified based on their inflammatory age levels; and can receive individualized interventions to reduce inflammatory age and improve clinical and immune responses to the therapeutic treatment (e.g., cancer immunotherapy, vaccination, anti-pathogen therapeutic).

An inflammatory age scoring system (iAge) can be used to classify subjects (e.g., cancer patients, vaccination subjects, subjects with an infectious disease) into those who have an immune system that can mount an effective response (e.g., mount an objective clinical response to immunotherapy, produce a protective response to a vaccine, or mount an immune response against a pathogen) versus those who will not. The inflammatory age scoring system can be used to guide initial therapy targeting inflammation to enable optimal objective responses in those patients who were classified as non-responders. The iAge can also be used to stratify subjects for different courses of vaccines or antipathogen therapy. A cytokine response score (CRS) can also be used to classify cancer patients into those who will mount an objective clinical response to immunotherapy versus those who will not.

Based on a subject's iAge, CRS, and/or Jak-STAT responses the subject can be classified as a responder or a nonresponder for the immunotherapy. Patients who are classified as nonresponders can be treated to lower their iAge, increase their CRS, and/or increase their Jak-STAT response so that the subject moves into a responder category. Classifications are made by comparing the subjects iAge, CRS, and/or Jak-STAT response to those of patients of similar chronological age. When a subject's iAge, CRS, and/or Jak-STAT response places them at a younger iAge for their age cohort, or a more responsive CRS and/or Jak-STAT score the subject can be a responder for immunotherapy. Subjects with older iAge for their age cohort, and/or lower scores for CRS and/or Jak-STAT can be treated to lower their iAge and/or increase their CRS and/or Jak-STAT score so that they move into a responder group for immunotherapy.

Based on a subject's iAge, CRS, Jak-STAT responses, cardiac age (cAge), and/or levels of MIG, LIF and/or SIRT3 the subject can be classified as high risk or low risk for cardiovascular disease. Patients who are classified as high risk can be treated to lower their iAge, increase their CRS, increase their Jak-STAT response, lower cAge, lower MIG, raise LIF and/or raise SIRT3 so that the subject moves into the low risk category. Classifications are made by comparing the subject's iAge, CRS, Jak-STAT responses, cAge, and/or levels of MIG, LIF and/or SIRT3 to those of patients of similar chronological age. When a subject's iAge, CRS, Jak-STAT responses, cAge, and/or levels of MIG, LIF and/or SIRT3 places them at a younger iAge for their age cohort, or a more responsive CRS and/or Jak-STAT score, a lower cAge, a lower MIG, a higher LIF, and/or a higher SIRT3 the subject is less at risk for cardiovascular disease. Subjects with older iAge for their age cohort, lower scores for CRS and/or Jak-STAT, older cAge, a higher MIG, a lower LIF, and/or a lower SIRT3 can be treated to lower their iAge, increase their CRS and/or Jak-STAT score, lower cAge, lower MIG, increase LIF, and/or increase SIRT3 so that they move into the lower risk cohort of patients.

A subject's MIG, LIF, and Sirtuin-3 levels can also be used to classify risk for cardiovascular disease. Patients can be classified by their levels of MIG, Sirtuin-3, LIF, and optionally other factors. For example, the patients can be assigned a cardiac age based on these factors with or without other factors. When a patient's levels of MIG, SIRT3, LIF, and/or cardiac age (cAge) places them in a younger quartile, quintile, decile (or other quantile) for their age cohort the subject is less at risk for cardiovascular disease. Subjects with older levels of MIG, SIRT3, LIF, and/or cAge for their age cohort can be treated to lower their levels of MIG, SIRT3, LIF, and/or cAge so that they move into the lower risk cohort of patients.

In an aspect, the disclosure describes diagnosing cardiovascular disease, monitoring cardiovascular disease progression, monitoring the treatment of cardiovascular disease, prognosing cardiovascular disease, treating cardiovascular disease, alleviating symptoms of cardiovascular disease, inhibiting progression of cardiovascular disease, and preventing cardiovascular disease, in a mammal using the markers, combinations of markers, treatments, prophylactic treatments, and/or agents provided herein.

In an aspect, the disclosure describes compounds and methods for modifying iAge (or cAge) of a subject. The iAge (or cAge) modification can reclassify the cohort of a subject undergoing cancer treatment, immunotherapy, or cardiovascular disease treatment. The compounds and methods can modify one or more markers involved in the iAge determination.

In an aspect, the disclosure describes compositions which can be used to improve the iAge of individuals within certain immunotypes. Treatments can include, for example, combinations of components that can alter the level of an iAge marker to healthier levels (lowers iAge) for one or more of the iAge markers: TRAIL, GroA, IFNg, MIG, or Eotaxin. Such combinations can include a combination of one or more of the following: iron bisglycinate, iron, biotin, caffeine, manganese chloride, niacin, carrageenan, beta-carotene, leutin, zinc-sulfate, vitamin D2, guar gum, kawain, L-methionine, indole-3-carbinol, and/or picetannol.

The disclosure also describes methods for identifying drugs, food compounds and other molecules that modify iAge (or cAge). These methods identify drugs, food compounds, and other molecules that interact with and modify the levels of certain markers involved with the iAge (or cAge) determination. These drugs, food compounds, and other molecules can be used with subjects to modify their iAge (or cAge) and so change the cohort in which the subject stratifies and so alter the response of the subject to treatment and/or risk of disease.

### BRIEF DESCRIPTION OF THE DRAWINGS

FIGS. 1A, 1B and 1C show graphs of iAge, naïve CD8(+) T-cells, and Jak STAT signaling responses.

FIG. 2 shows the stratification of cancer patients by iAge and CRS into responders and nonresponders.

FIG. 3 shows the stratification of cancer patients using iAge.

FIG. 4 depicts the fold change in MIG mRNA and SIRT3 mRNA after passage of cells following differentiation into endothelial cells from hiPSCs.

FIG. 5 is a bar graph showing the relative expression levels of CXCR3 in different cell types.

FIG. 6 shows the four immunotypes of the super-healthy group of patients.

FIG. 7 shows the six immunotypes of the normal group of patients.

FIG. 8 shows the average levels of Eotaxin, TRAIL, GroA, IFNg, and MIG (after normalization of the data as discussed below) for each the super-healthy immunotypes (SH1-4).

FIG. 9 shows the average levels of Eotaxin, TRAIL, GroA, IFNg, and MIG (after normalization of the data as discussed below) for each the normal immunotypes (N1-6).

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, “activation” is defined to be a physiological condition upon exposure to a substance, allergen, drug, protein, chemical, or other stimulus, or upon removal of a substance, allergen, drug, protein, chemical or other stimulus.

As used herein, an “antibody” is defined to be a protein functionally defined as a ligand-binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either V_{L} or V_{H}), camelid VHH domains, and multi-specific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, an “antigen” is defined to be a molecule that provokes an immune response. This immune response may involve either antibody production, or the activation of specific immunologically-competent cells, or both. The skilled artisan will understand that any macromolecule, including, but not limited to, virtually all proteins or peptides, including glycosylated polypeptides, phosphorylated polypeptides, and other post-translation modified polypeptides including polypeptides modified with lipids, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or can be a macromolecule besides a polypeptide. Such a biological sample can include, but is not limited to a tissue sample, a tumor sample, a cell or a fluid with other biological components.

As used herein, the terms “Chimeric Antigen Receptor” and the term “CAR” are used interchangeably. As used herein, a “CAR” is defined to be a fusion protein comprising antigen recognition moieties and cell-activation elements.

As used herein, a “CAR T-cell” or “CAR T-lymphocyte” are used interchangeably, and are defined to be a T-cell containing the capability of producing CAR polypeptide, regardless of actual expression level. For example a cell that is capable of expressing a CAR is a T-cell containing nucleic acid sequences for the expression of the CAR in the cell.

As used herein, an “effective amount” or “therapeutically effective amount” are used interchangeably, and defined to be an amount of a compound, formulation, material, or composition, as described herein effective to achieve a particular biological result.

As used herein, an “epitope” is defined to be the portion of an antigen capable of eliciting an immune response, or the portion of an antigen that binds to an antibody. Epitopes can be a protein sequence or subsequence that is recognized by an antibody.

As used herein, an “expression vector” and an “expression construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrated into the chromosome. When integrated into the chromosome the nucleic acids comprising the expression vector or expression construct will be an expression vector or expression construct.

As used herein, “heart failure” often called congestive heart failure (CHF) or congestive cardiac failure (CCF), means a condition that occurs when the heart is unable to provide sufficient pump action to maintain blood flow to meet the needs of the body. Heart failure can cause a number of symptoms including shortness of breath, leg swelling, and exercise intolerance. The condition is typically diagnosed by patient physical examination and confirmed with echocardiography. Common causes of heart failure include myocardial infarction and other forms of ischemic heart disease, hypertension, valvular heart disease, and cardiomyopathy. The term heart failure is sometimes incorrectly used for other cardiac-related illnesses, such as myocardial infarction (heart attack) or cardiac arrest, which can cause heart failure but are not equivalent to heart failure.

As used herein, “heterologous” is defined to mean the nucleic acid and/or polypeptide is not homologous to the host cell. Alternatively, “heterologous” means that portions of a nucleic acid or polypeptide that are joined together to make a combination where the portions are from different species, and the combination is not found in nature.

As used herein, the term “impaired immune function” is defined to be any reduction in immune function in an individual, as compared to a fully healthy individual. Individuals with an impaired immune function are readily identifiable by substantially increased abundance of CD8+ CD28-cells or more broadly by reduced cytokine responses, increased baseline phosphoprotein levels and other co-occurring measure.

As used herein, the term “inflammasome” is defined as cytosolic multiprotein complexes that are composed of an inflammasome-initiating sensor, apoptosis-associated speck-like protein containing a CARD (Caspase Activation and Recruitment Domain) acts as an adaptor protein and the protease-caspase-1. Inflammasome-initiating sensors include members of the NLRs the pyrin and HIN domain-containing (also known as PYHIN, Aim 2-like receptors, or ALRs; e.g., Aim2), or the TRIM (e.g., pyrin) family. Complex assembly leads to caspase-l-dependent cleavage of cytokines pro-interleukin 1α (pro-IL-1β) and pro-IL-18 into secreted mature forms. In addition, inflammasomes initiate pyroptotic cell death.

As used herein, a “single chain antibody” (scFv) is defined as an immunoglobulin molecule with function in antigen-binding activities. An antibody in scFv (single chain fragment variable) format consists of variable regions of heavy (V_{H}) and light (V_{L}) chains, which are joined together by a flexible peptide linker.

### Immunological Age

The Jak/STAT signaling pathway is critical for meeting the multiple challenges encountered by the immune system, from fighting infections to maintaining immune tolerance. Clearly STATs are also involved in the development and function of the immune system in humans and play a key role in maintaining immune surveillance of cancer (Nature. 2007; 450(7171):903-7; Nat Rev Cancer (2009) 9:798-809).

The Jak-STAT pathway can be profoundly altered with aging and this is one major cause of immune dysfunction in older adults. A cytokine response score (CRS) can be used to predict immune decline and reduction in immune surveillance of cancer.

An inflammatory age scoring system (iAge) can also be used to predict age-associated multimorbidity and mortality. iAge can be extremely sensitive as a biomarker of cardiovascular health since elevated levels predict left ventricular remodeling and arterial stiffness even in very healthy older subject with no clinical or laboratory cardiovascular risk factors. iAge can also identify subclinical immunodeficient young patients (10% of subjects 16-35 years old) who cannot mount responses to any strain of the influenza vaccine in any of the years studied (up to 6 years follow-up). These subjects are characterized by having an older-like immunological phenotype with regards to their immune cell composition, ex vivo responses to multiple acute stimuli, and expression of gene modules associated with advanced age.

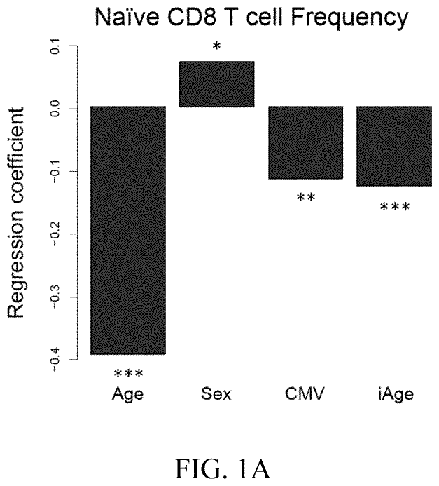

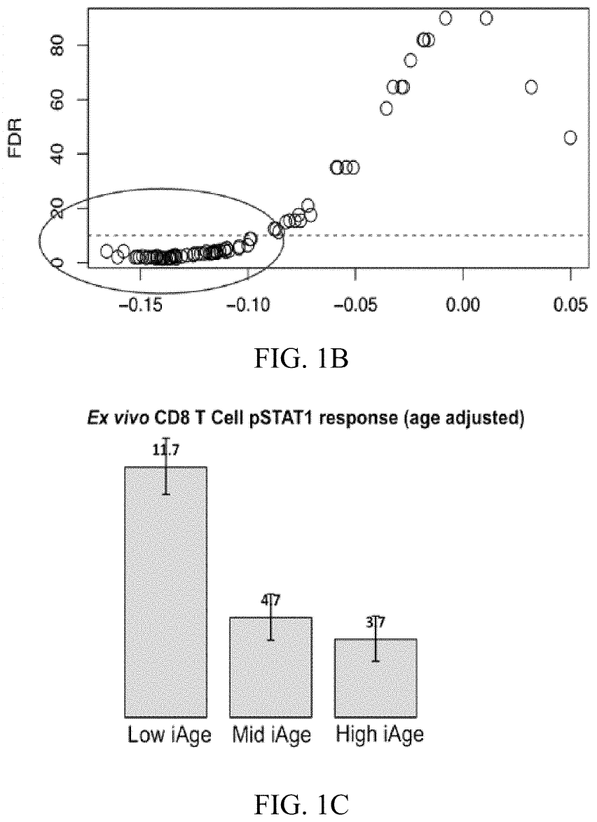

Since the cytokine response score CRS and iAge are independent measures of inflammation, diminished Jak-STAT signaling pathway in T cells, and low naïve CD8(+) T cell counts (FIG. 1A-C) these measures can be used to stratify cancer patients with respect to their clinical responses to immunotherapy. The methods described herein use blood inflammatory markers CRS and iAge to stratify cancer patients into responder and nonresponders groups for immunotherapy. The nonresponders can be treated to reduce their iAge and/or increase their CRS (and/or Jak-STAT score) so that the nonresponders obtain iAge and/or CRS (and/or Jak-STAT score) that places them into a responder group.

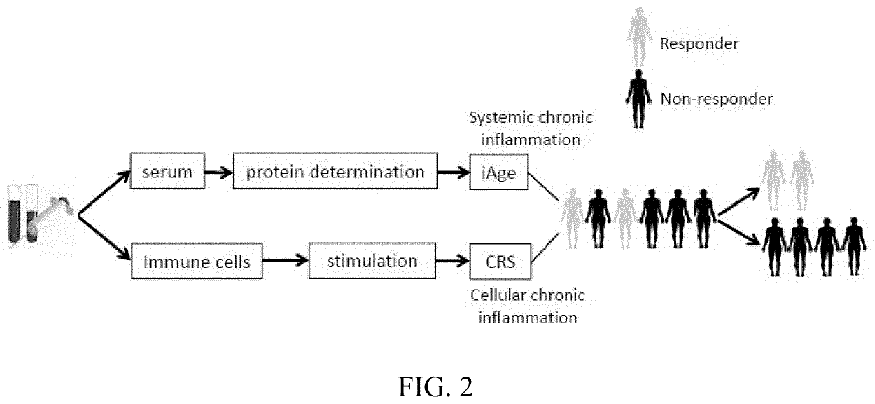

The procedure involves the extraction of peripheral blood samples by venipuncture, or by any appropriate method, from candidate cancer patients prior to infusion with immunotherapy treatment (FIG. 2). Immunotherapy treatment may comprise the use of certain molecules including antibodies, small molecules, etc. against inhibitory immune receptors. Blood serum is separated from blood cells by centrifugation of clotted blood, or by any other appropriate method (FIG. 2).

Construction of iAge: For serum protein determination, the resulting sera can be mixed with antibody-linked magnetic beads on 96-well filter-bottom plates and can be incubated at room temperature for 2 h followed by overnight incubation at 4° C. Room temperature incubation steps can be performed on an orbital shaker at 500-600 rpm. Plates can be vacuum filtered and washed twice with wash buffer, then incubated with biotinylated detection antibody for 2 h at room temperature. Samples can be then filtered and washed twice as above and re-suspended in streptavidin-PE. After incubation for 40 minutes at room temperature, two additional vacuum washes can be performed, and the samples can be re-suspended in Reading Buffer. Each sample can be measured in duplicate or triplicate. Plates can be read using a Luminex 200 instrument with a lower bound of 100 beads per sample per cytokine and mean fluorescence intensity (MFI) is recorded.

To derive inflammatory age (iAge) (FIG. 2), the mean fluorescence intensity can be normalized and used for multiple regression analysis, which is computed using the following regression coefficients: MIG: 0.6357, TRAIL: −0.3760, IFNG: −0.3235, EOTAXIN: 0.2912, GROA: −0.2723, IL2: −0.2063, TGFA: −0.1978, PAI1: −0.1587, LIF: −0.1587, LEPTIN: 0.1549, MIP1A: 0.1547, IL1B: 0.1471. The MFI can be multiplied by the regression coefficient for the protein, and these numbers can be all added together to give the iAge of the subject. Table 1 below lists the ranges of iAge within chronological age decades.

TABLE 1
iAge Ranges

| Chronological Age (years) | iAge Range |
|---|---|
| 10-19 | 18.1-58.3 |
| 20-29 | 18.5-78.9 |
| 30-39 | 16.9-70.2 |
| 40-49 | 21.5-74.1 |
| 50-59 | 23.1-74.4 |
| 60-69 | 28.1-76.6 |
| 70-79 | 35.6-77.6 |
| 80-96 | 37.9-78.5 |

Those markers with positive regression coefficients increased in serum concentration with age (MIG, EOTAXIN, LEPTIN, MIP1A, and IL1B) and those with negative regression coefficients decreased in serum concentration with age (TRAIL, IFNG, GROA, IL2, TGFA, PAI1, and LIF).

MIG (monokine induced by gamma interferon) is a small cytokine belonging to the CXC chemokine family. MIG is one of the chemokines which plays a role to induce chemotaxis, promote differentiation and multiplication of leukocytes, and cause tissue extravasation. MIG regulates immune cell migration, differentiation, and activation. Tumor-infiltrating lymphocytes are a key for clinical outcomes and prediction of the response to checkpoint inhibitors. In vivo studies suggest the axis plays a tumorigenic role by increasing tumor proliferation and metastasis. MIG predominantly mediates lymphocytic infiltration to the focal sites and suppresses tumor growth. MIG binds to C-X-C motif chemokine 3 of the CXCR3 receptor.

TRAIL (TNF-related apoptosis-inducing ligand) is a cytokine that is produced and secreted by most normal tissue cells. It is thought to cause apoptosis primarily in tumor cells by binding to certain death receptors. TRAIL has also been designated CD253 (cluster of differentiation 253) and TNFSF-10 (tumor necrosis factor (ligand) superfamily, member 10). TRAIL is described in Wiley et al Immunity 1005 3: 673-82 as well as Pitti J. Biol. Chem. 1996 271 : 12687-90.

IFNG (otherwise known as interferon gamma, IFNy or type II interferon) is a dimerized soluble cytokine that is the only member of the type II class of interferons. IFNG is critical for innate and adaptive immunity against viral, some bacterial and protozoan infections. IFNG is an important activator of macrophages and inducer of Class II major histocompatibility complex (MHC) molecule expression. IFNG is described In Schoenborn et al Adv. Immunol. 2007 96: 4I-IOI as well as Gray Nature. I982 298: 859-63.

Eotaxin (also known as C-C motif chemokine I I or eosinophil chemotactic protein) is a small cytokine belonging to the CC chemokine family. Eotaxin selectively recruits eosinophils by inducing their chemotaxis, and therefore, is implicated in allergic responses. The effects of eotaxin is mediated by its binding to a G-protein-linked receptor known as a chemokine receptor. Chemokine receptors for which CCLI I is a ligand include CCR2, CCR3 and CCR5. Eotaxin is described in Kitaura et al The Journal of Biological Chemistry I 996 27I: 7725-30 and Jose et al The Journal of Experimental Medicine I994 I 79: 88I-7.

GROA (also known as CXCLI, the GROI oncogene, GROa, KC, neutrophil activating protein 3 (NAP-3) and melanoma growth stimulating activity, alpha (MSGA-a)) is secreted by human melanoma cells, has mitogenic properties and is implicated in melanoma pathogenesis. GROA is expressed by macrophages, neutrophils and epithelial cells, and has neutrophil chemoattractant activity. This chemokine elicits its effects by signaling through the chemokine receptor CXCR2. GROA is described in Haskill et al Proc. Natl. Acad. Sci. U.S.A. I90 87 (I9): 7732-6.

IL-2 is one of the key cytokines with pleiotropic effects on the immune system. It is a 15.5-16 kDa protein that regulates the activities of white blood cells (leukocytes, often lymphocytes) that are responsible for immunity. The major sources of IL-2 are activated CD4+ T cells, activated CD8+ T cells, NK cells, dendritic cells and macrophages. IL-2 is an important factor for the maintenance of CD4+ regulatory T cells and plays a critical role in the differentiation of CD4+ T cells into a variety of subsets. It can promote CD8+ T-cell and NK cell cytotoxicity activity, and modulate T-cell differentiation programs in response to antigen, promoting naive CD4+ T-cell differentiation into T helper-1 (Th1) and T helper-2 (Th2) cells while inhibiting T helper-17 (Th17) differentiation.

TGFA (transforming growth factor alpha) is a polypeptide of 5.7 kDa that is partially homologous to EGF. TGFA is a growth factor that is a ligand for the epidermal growth factor receptor, which activates a signaling pathway for cell proliferation, differentiation and development. TGFA also is a potent stimulator of cell migration. TGFA can be produced in macrophages, brain cells, and keratinocytes. TGFA can induce epithelial development. TGFA can also upregulate TLR expression and function augmenting host cell defense mechanisms at epithelial surfaces. TGFA may act as either a transmembrane-bound ligand or a soluble ligand. TGFA has been associated with many types of cancers, and it may also be involved in some cases of cleft lip/palate. Alternatively spliced transcript variants encoding different isoforms have been found for this gene.

PAI1 (plasminogen activator inhibitor-1) is a member of the serine proteinase inhibitor (serpin) superfamily. PAI1 is the principal inhibitor of tissue plasminogen activator (tPA) and urokinase (uPA), and hence is an inhibitor of fibrinolysis. PAI1 is also a regulator of cell migration. PAI1 can play a role in a number of age-related, conditions including, for example, inflammation, atherosclerosis, insulin resistance, obesity, comorbidities, and Werner syndrome. PAI1 can play a host protective role during the acute phase of infection by regulating interferon gamma release. IFNG regulates PAI-1 expression, which suggests an intricate interplay between PAI-1 and IFNG. PAI1 can also activate macrophages through Toll-like receptor 4 (TLR4) and can promote migration of pro-cancer M2 macrophages into tumors.

LIF (leukemia inhibitory factor) is interleukin 6 class cytokine with pleiotropic effects impacting several different systems. When LIF levels drop, cells differentiate. LIF has the capacity to induce terminal differentiation in leukemic cells. Its activities include the induction of hematopoietic differentiation in normal and myeloid leukemia cells, the induction of neuronal cell differentiation, and the stimulation of acute-phase protein synthesis in hepatocytes. The protein encoded by this gene is a pleiotropic cytokine with roles in several different systems. It is involved in the induction of hematopoietic differentiation in normal and myeloid leukemia cells, induction of neuronal cell differentiation, regulator of mesenchymal to epithelial conversion during kidney development, and may also have a role in immune tolerance at the maternal-fetal interface. Alternatively spliced transcript variants encoding multiple isoforms have been observed for this gene. LIF functions through both autocrine and paracrine manners. LIF binds to its specific receptor LIFR, then recruits gp130 to form a high affinity receptor complex to induce the activation of the downstream signal pathways including JAK/STAT3, PI3K/AKT, ERK1/2 and mTOR signaling. Further studies have clearly proven that LIF is a multifunctional protein which has a broad biological functions in neuronal, hepatic, endocrine, inflammatory and immune systems. LIF regulates the embryonic stem cell self-renewal and is an indispensable factor to maintain mouse embryonic stem cell pluripotency. The expression of LIF is induced under inflammatory stress as an anti-inflammatory agent.

LEPTIN is secreted by white adipocytes into the circulation and plays a major role in the regulation of energy homeostasis. LEPTIN binds to the leptin receptor in the brain, which activates downstream signaling pathways that inhibit feeding and promote energy expenditure. LEPTIN also has several endocrine functions, and is involved in the regulation of immune and inflammatory responses, hematopoiesis, angiogenesis, reproduction, bone formation and wound healing. LEPTIN can directly link nutritional status and pro-inflammatory T helper 1 immune responses, and a decrease of LEPTIN plasma concentration during food deprivation can lead to an impaired immune function. LEPTIN is associated with the pathogenesis of chronic inflammation, and elevated circulating LEPTIN levels in obesity appear to contribute to low-grade inflammation which makes obese individuals more susceptible to increased risk of developing cardiovascular diseases, type II diabetes, and degenerative disease including autoimmunity and cancer. Reduced levels of LEPTIN such as those found in malnourished individuals have been linked to increased risk of infection and reduced cell-mediated immune responses. Mutations in this gene and its regulatory regions cause severe obesity and morbid obesity with hypogonadism in human patients. A mutation in this gene has also been linked to type 2 diabetes mellitus development.

MIP1A (macrophage inflammatory protein) is a member of the CC or beta chemokine subfamily. MIP1A regulates leukocyte activation and trafficking. MIP1A acts as a chemoattractant to a variety of cells including monocytes, T cells, B cells and eosinophils. MIP1A plays a role in inflammatory responses through binding to the receptors CCR1, CCR4 and CCR5.

IL-1B (Interleukin-1 beta) is a member of the interleukin 1 cytokine family. IL-1B is an important mediator of the inflammatory response, and is involved in a variety of cellular activities, including cell proliferation, differentiation, and apoptosis. LI-1B is produced by activated macrophages as a proprotein, which is proteolytically processed to its active form by caspase 1 (CASP1/ICE).

iAge predicts pulse-wave velocity (a measure of arterial stiffness, or the rate at which pressure waves move down the vessel) which correlates with cardiovascular health.

Construction of CRS: Separation of immune cells may comprise the use of differential centrifugation of blood by density gradient (FIG. 2). The resulting cell pellet can be suspended in warm media, wash twice and resuspended at 0.5×10\hat{ }6 viable cells/mL. 200 uL of cell sample can be plated per well in 96-well deep-well plates. After resting for 1 hour at 37° C., cells can be stimulated by adding 50 ul of cytokine (IFNa, IFNg, IL-6, IL-7, IL-10, IL-2, or IL-21) (FIG. 2) and incubated at 37° C. for 15 minutes. The cells can be fixed with paraformaldehyde, permeabilized with methanol, and kept at −80 C overnight. Each well can then be bar-coded using a combination of Pacific Orange and Alexa-750 dyes (Invitrogen, Carlsbad, Calif.) and pooled in tubes. The cells can be washed with FACS buffer (PBS supplemented with 2% FBS and 0.1% soium azide), and stained with the following antibodies (all from BD Biosciences, San Jose, Calif.): CD3 Pacific Blue, CD4 PerCP-Cy5.5, CD20 PerCp-Cy5.5, CD33 PE-Cy7, CD45RA Qdot 605, pSTAT-1 AlexaFluor488, pSTAT-3 AlexaFluor647, pSTAT-5 PE. The samples can be washed and resuspended in FACS buffer. 100,000 cells per stimulation condition are collected using DIVA 6.0 software on an LSRII flow cytometer (BD Biosciences). Data analysis can be performed using FlowJo v9.3 by gating on live cells based on forward versus side scatter profiles, then on singlets using forward scatter area versus height, followed by cell subset-specific gating.

Fold-change difference due to stimulation can be computed as the ratio of the cell, cytokine stimulation, phosphoprotein measure to the raw, un-normalized, cell-phosphoprotein matching baseline that was measured on the same plate. The data can be normalized by scaling individuals by the average of the assay on the day in which they were measured.

To construct the Cytokine Response Score (CRS) (FIG. 2) 15 reproducible age-associated normalized cytokine responses can be expressed as fold increases over baseline (unstimulated) and the fold increases for the following can be summed: CD8+ cells, stimulate with IFNa and measure pSTAT1, 3 and 5; CD8+ cells, stimulate with IL6 and measure pSTAT1, 3 and 5, CD8+ cells, stimulate with IFNg and measure pSTAT1, CD8+ cells, stimulate with IL21 and measure pSTAT1; CD4+ cells, stimulate with IFNa and measure pSTAT5, CD4+ cells, stimulate with IL6 and measure pSTAT5, CD20+ cells, stimulate with IFNa and measure pSTAT1, Monocytes stimulate with IL10 and measure pSTAT3, Monocytes stimulate with IFNg and measure pSTAT3, Monocytes stimulate with IFNa and measure pSTAT3, and Monocytes stimulate with IL6 and measure pSTAT3.

IFNA (Interferon alpha) is a member of the type I interferon class. And has thirteen (13) variants in humans. IFNA is secreted by hematopoietic cells, predominately plasmacytoid dendritic cells. IFNA can have either protective or deleterious roles. IFNA can be induced by ssRNA, dsRNA, and cytosolic DNA from viruses or bacteria. IFNA can induce caspase-11 expression, which contributes to activation of non-canonical inflammasome. Use of recombinant IFNA has been shown to be effective in reducing the symptoms and duration of the common cold.

IFNG (Interferon gamma) is a member of the type II interferon class. The encoded protein is secreted by cells of both the innate and adaptive immune systems. The active protein is a homodimer that binds to the interferon gamma receptor which triggers a cellular response to viral and microbial infections. Mutations in this gene are associated with an increased susceptibility to viral, bacterial and parasitic infections and to several autoimmune diseases.

IL6 is a cytokine with pleiotropic effects on inflammation, immune response, and hematopoiesis. IL6 is promptly and transiently produced in response to infections and tissue injuries, contributes to host defense through the stimulation of acute phase responses, hematopoiesis, and immune reactions. IL6 functions in inflammation and the maturation of B cells. In addition, IL6 has been shown to be an endogenous pyrogen capable of inducing fever in people with autoimmune diseases or infections. IL6 is primarily produced at sites of acute and chronic inflammation, where it is secreted into the serum and induces a transcriptional inflammatory response through interleukin 6 receptor, alpha. IL6 is implicated in a wide variety of inflammation-associated disease states, including susceptibility to diabetes mellitus and systemic juvenile rheumatoid arthritis. Dysregulated, continual synthesis of IL-6 plays a pathological effect on chronic inflammation and autoimmunity. Alternative splicing results in multiple transcript variants.

IL10 is a cytokine with pleiotropic effects in immunoregulation and inflammation. IL-10 is an anti-inflammatory cytokine and during infection it inhibits the activity of Th1 cells, NK cells, and macrophages, all of which are required for optimal pathogen clearance but also contribute to tissue damage. IL10 can directly regulate innate and adaptive Th1 and Th2 responses by limiting T cell activation and differentiation in the lymph nodes as well as suppressing proinflammatory responses in tissues. It also enhances B cell survival, proliferation, and antibody production. This cytokine can block NF-kappa B activity, and is involved in the regulation of the JAK-STAT signaling pathway. Knockout studies in mice suggested the function of this cytokine as an essential immunoregulator in the intestinal tract.

IL21 is a member of the common-gamma chain family of cytokines with immunoregulatory activity. IL21 plays a role in both the innate and adaptive immune responses by inducing the differentiation, proliferation and activity of multiple target cells including macrophages, natural killer cells, B cells, cytotoxic T cells, and epithelial cells. IL21 is important to anti-tumor and antiviral responses and also exerts major effects on inflammatory responses that promote the development of autoimmune diseases and inflammatory disorders.

pSTAT1 (phosphorylated signal transducer and activator of transcription 1) mediates cellular responses to interferons (IFNs), cytokine KITLG/SCF and other cytokines and other growth factors. Following type I IFN (IFN-alpha and IFN-beta) binding to cell surface receptors, signaling via protein kinases leads to activation of Jak kinases (TYK2 and JAK1) and to tyrosine phosphorylation of STAT1 and STAT2. The phosphorylated STATs dimerize and associate with ISGF3G/IRF-9 to form a complex termed ISGF3 transcription factor, that enters the nucleus (PubMed:28753426). ISGF3 binds to the IFN stimulated response element (ISRE) to activate the transcription of IFN-stimulated genes (ISG), which drive the cell in an antiviral state. In response to type II IFN (IFN-gamma), STAT1 is tyrosine- and serine-phosphorylated (PubMed:26479788). It then forms a homodimer termed IFN-gamma-activated factor (GAF), migrates into the nucleus and binds to the IFN gamma activated sequence (GAS) to drive the expression of the target genes, inducing a cellular antiviral state.

pSTAT 3 (phosphorylated signal transducer and activator of transcription 3) mediates cellular responses to interleukins, KITLG/SCF, LEP and other growth factors. Once activated, recruits coactivators, such as NCOA1 or MED1, to the promoter region of the target gene. Binds to the interleukin-6 (IL-6)-responsive elements identified in the promoters of various acute-phase protein genes. Activated by IL31 through IL31RA. Acts as a regulator of inflammatory response by regulating differentiation of naive CD4+ T-cells into T-helper Th17 or regulatory T-cells (Treg): deacetylation and oxidation of lysine residues by LOXL3, disrupts STAT3 dimerization and inhibits its transcription activity.

pSTAT 5 (phosphorylated signal transducer and activator of transcription 5) is activated by Janus-activated kinases (JAK) downstream of cytokine receptors. STAT5 proteins are activated by a wide variety of hematopoietic and nonhematopoietic cytokines and growth factors, all of which use the JAK-STAT signaling pathway as their main mode of signal transduction. STAT5 proteins critically regulate vital cellular functions such as proliferation, differentiation, and survival. STAT5 plays an important role in the maintenance of normal immune function and homeostasis, both of which are regulated by specific members of IL-2 family of cytokines, which share a common gamma chain (γ(c)) in their receptor complex. STAT5 critically mediates the biological actions of members of the γ(c) family of cytokines in the immune system. Essentially, STAT5 plays a critical role in the function and development of Tregs, and consistently activated STAT5 is associated with a suppression in antitumor immunity and an increase in proliferation, invasion, and survival of tumor cells.

### Immunotypes

Five markers which are significant contributors to iAge were used in deriving immunotypes. The five markers are Eotaxin, GroA, INFg, MIG, and TRAIL. Using information from 1,642 samples, the levels of these markers were subjected to a Principal Component Analysis. In a PCA, the levels of the five (5) markers can be standardized so that each contributes equally. This standardized data can then be subject to a covariance matrix computation to see if some of the variables are behaving in a correlated fashion. Eigenvectors and Eigenvalues can be calculated for the covariance matrix to identify the Principle Components. Principal components are new variables that are constructed as linear combinations or mixtures of the initial variables. Principal Components represent the directions of the data that explain a maximal amount of variance, that is to say, the lines that capture most information of the data. Components of lesser value can be discarded and more significant components can be kept (reducing the dimensionality of the data set). This data can be recast along the Principal Component axes.

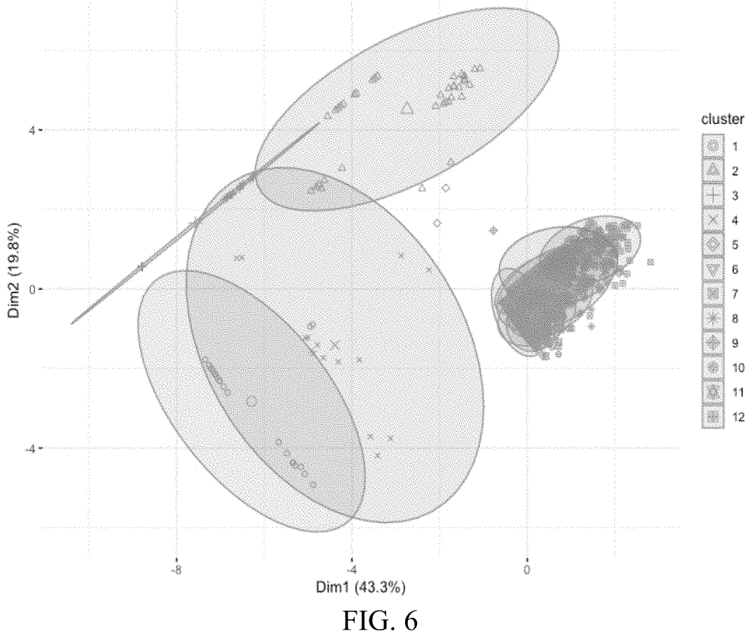

The PCA of the patient data produced two groups: a super-healthy group and a normal health group. The super healthy group was divided into four immunotypes 1-4 which are shown in FIG. 6. In FIG. 6, the level of each protein marker was standardized (50 percentile=0 with positive values indicating a higher contribution to iAge) and minus 4 was the level of marker in patients with an iAge 20 years younger than their chronological age (lower/younger iAge correlates with better health outcomes). Immunotype SH1 had low iAge score levels for Eotaxin, GroA, IFNg, and MIG (indicating a lower contribution to iAge), but high iAge score levels of TRAIL (indicating a higher contribution to iAge). Immunotype SH2 had low iAge scores for Eotaxin and TRAIL, high iAge score levels of GroA and INFg, and moderate iAge score levels for MIG. Immunotype SH3 had low iAge score levels for all five markers (Eotaxin, GroA, IFNg, MIG, TRAIL). Immunotype SH4 had low iAge scores for GroA, IFNg, and TRAIL, high iAge score levels of MIG, and moderate iAge score levels for Eotaxin.

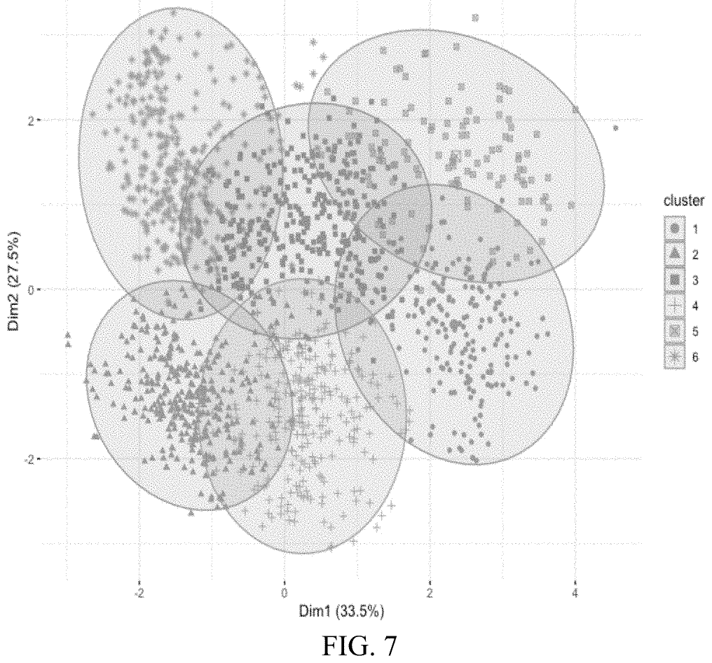

The normal group was divided into six Immunotypes 1-6 which are shown in FIG. 7. In FIG. 7, the level of each protein marker was standardized (50 percentile=0) and minus 4 was the level of marker in patients with an iAge 20 years younger than their chronological age. In addition, two additional components were added for the PCA of the normal group: number of markers in the minus direction (low iAge), and the difference between iAge and chronological age. Immunotype N1 had negative iAge score levels for Eotaxin and TRAIL, and high iAge score levels for GroA, IFNg and MIG. Immunotype N2 had moderate iAge score levels for Eotaxin, GroA, IFNg, and MIG, and high iAge score levels for TRAIL. Immunotype N3 had moderate iAge score levels for MIG and TRAIL, and high iAge score levels for Eotaxin, GroA, and IFNg. Immunotype N4 had moderate iAge score levels for Eotaxin and MIG, and high iAge score levels for GroA, IFNg, and TRAIL. Immunotype N4 had moderate iAge score levels for MIG and TRAIL, and high iAge score levels for Eotaxin, GroA, and IFNg. Immunotype N5 had low iAge score levels for TRAIL, and high iAge score levels for GroA, IFNg, MIG, and TRAIL. Immunotype N6 had moderate iAge score levels for GroA and IFNg, and high iAge score levels for Eotaxin, MIG, and TRAIL.

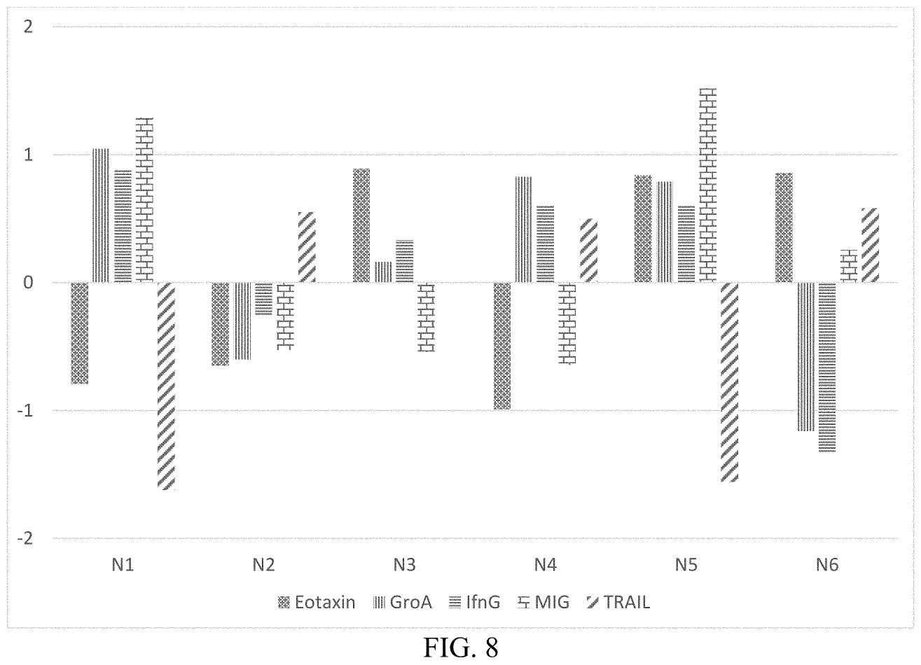

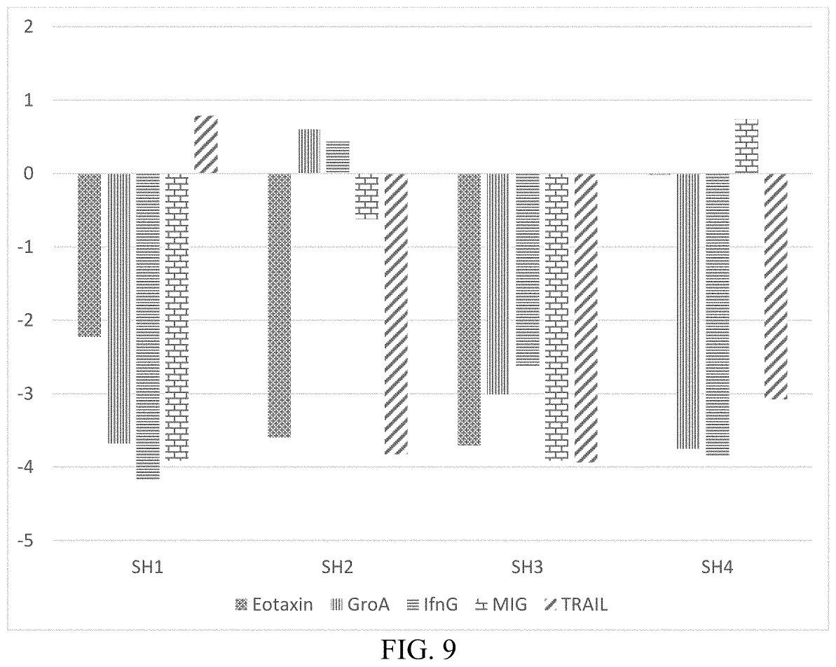

FIGS. 8 and 9 show that each of the ten immunotypes has a different signature for the five markers (different levels of the five markers after the markers are normalized as discussed above, i.e., normalized so zero=50^{th} percentile and 4=level in patients with an iAge 20 years younger than their chronological age). Thus, a common treatment of each immunotype can be provided to members of the immunotype to lower the iAge of any member of the immunotype which will lower iAge and improve the patients health, well-being and longevity.

Construction of Cardiac Age.

To derive Cardiac age (cAge), patient samples are obtained and processed similar to the description above for iAge. The mean fluorescence intensity can be normalized and used for multiple regression analysis. The levels of MIG, LIF and SIRT3 are diagnostic for risk of cardiovascular health. Other parameters that can be used to compute cardiac age include, for example, aortic pulse wave velocity, a measure of vascular stiffness; relative wall thickness (RWT), a measure of ventricular remodeling, and early diastolic mitral annular velocities (e′), a measure of ventricular relaxation. Still other parameters include, for example, sex, BMI, heart rate, systolic blood pressure, fasting glucose and total cholesterol to HDL ratio. The levels of MIG, LIF, SIRT3, and/or other measurements for a subject can be compared to those of other subjects of the same age and/or different ages to determine the quantile of the subject for each factor, or for the factors of subjects of different ages. Low quantile rank for MIG is diagnostic for low risk of cardiovascular disease, and high quantile rank for MIG is diagnostic for a higher risk of cardiovascular disease. High quantile rank for LIF and/or SIRT3 are diagnostic for low risk of cardiovascular disease, and high quantile rank for LIF and/or SIRT3 are diagnostic for a higher risk of cardiovascular disease. Other parameters (factors) can also be included in the analysis and, for example, high quantile rank for pulse wave velocity are diagnostic for a higher risk of cardiovascular disease, high quantile rank for abnormal RWT are diagnostic for a higher risk of cardiovascular disease, and lower quantile early diastolic mitral annular velocities are also diagnostic for higher risk of cardiovascular disease. Multiple parameters and/or factors can be combined to compute cardiac age, for example, MIG, LIF and SIRT3 can be used to derive cardiac age, or these factors can be combined with other parameters (e.g., aortic pulse wave velocity, RWT, and/or early diastolic mitral annular velocities) to derive cardiac age. When these factors and/or parameters are combined, high quantile rank will correlate with older cardiac age and a higher risk of cardiovascular disease, and a low quantile rank will correlate with younger cardiac age and a lower risk of cardiovascular disease. Quantile ranks can include, for example, quartiles, quintiles or deciles.

SIRT3 (Situin-3, a NAD-dependent deacetylase) is member of the mammalian sirtuin family of proteins, which are homologs to the yeast Sir2 protein. SIRT3 exhibits NAD+-dependent deacetylase activity. SIRT3 is a regulator of the mitochondrial adaptive response to stress, such as metabolic reprogramming and antioxidant defense mechanisms. SIRT3 mediates cellular resistance toward various forms of stress by maintaining genomic stability and mitochondrial integrity. SIRT3 is central to the maintenance of appropriate mitochondrial function by limiting oxidative stress, and reducing reactive oxygen species (ROS) production with a decrease in mitochondrial membrane potential. SIRT3 has cardio-protective properties involved in mitochondrial homeostasis, stem cell and tissue maintenance during aging, and linked to the beneficial effects of diet, caloric restriction and exercise in maintaining cardiovascular health and longevity.

MIG positively correlates with cardiovascular aging markers PWV (R=0.22), a measure of arterial stiffness, and RWT (R=0.3), a measure of cardiac remodeling; and a negative correlation between LIF and PWV (R=−0.27), and RWT (R=−0.22). Subclinical cardiac tissue remodeling and increased arterial stiffness can be found in otherwise healthy individuals with elevated levels of MIG and low levels of LIF.

Patients with subclinical cardiac tissue remodeling and increased arterial stiffness can be otherwise healthy individuals who have elevated levels of MIG and low levels of SIRT-3 and LIF. Cardiac tissue remodeling and increased arterial stiffness are risk factors associated with poorer outcomes in cardiovascular disease. The largest contributor to the inflammatory clock, MIG, was positively correlated with subclinical levels of arterial stiffness and cardiac remodeling even in healthy older adults with no clinical or laboratory evidence of cardiovascular disease. The inflammatory clock (iAge) can also be used as an early molecular marker for cardiovascular malfunctioning.

At least two sources of MIG-mediated inflammation can ensue with aging based on our findings; one that is age-intrinsic and observed in aging endothelia, and one independent of age (likely as a response to cumulative exposure to environmental insults). In contrast, there was no significant correlation between known disease risk factors (BMI, smoking, dyslipidemia) and the levels of MIG gene or protein expression. MIG overproduction can be caused by cellular aging per-se, which triggers metabolic dysfunction with production of damage-associated molecular patterns (DAMPs) such as adenine and N4-acetylcytidine. These DAMPs can then act through the inflammasome machinery, such as NLRC4, to regulate multiple inflammatory signals including IL-1β and MIG.

Endothelium has a critical role in the etiology of hypertension and arterial stiffness, and more advanced signs of cardiovascular aging such as tissue remodeling and cardiac hypertrophy are often preceded and may be initiated by malfunctioning of aged endothelia. Endothelial cells show a time-dependent increase in MIG transcript levels, which was concomitant with a drop in SIRT3 expression, and with a decrease in the number of vascular networks formed by the endothelial cells. Young endothelia is a target of MIG from other sources, and MIG can down-regulate SIRT3 expression in the endothelial cells. In addition, endothelia cells made from hiPSC (human, induced pluripotent stem cells) but not cardiomyocytes made from hiPSC, express CXCR3 he receptor for MIG. MIG can act both in a paracrine fashion, wherein increasing levels of this chemokine from immune sources affect endothelial cell function, and in an autocrine fashion on endothelial cells likely producing a positive feedback loop where increasing doses of MIG and expression of its receptor in these cells leads to cumulative deterioration of endothelial function in aging. Exposure of endothelial cells to MIG can also reduce the endothelial cell's capacity for forming tubular networks, and MIG can reduce vasorelaxation in the aorta.

### Immunosenescence

The immune system undergoes marked shifts in composition and function with aging, a pattern of changes that are together termed “immunosenescence”.

Immunosenescence impacts both the innate and adaptive arms of the immune system and major features of immunosenescence include alteration in immune cell subset frequencies, defective antigen presentation, reduced cytotoxic function, and restricted T cell repertoire (Pawelec G, Larbi A. (2008), Immunity and ageing in man: Annual Review 2006/2007. Exper Gerontol 43:34-38; Weiskopf D, Weinberger B, GrubeckLoebenstein B. (2009), The aging of the immune system. Transpl Int. 22:1041-1050, both of which are incorporated by reference in their entirety for all purposes).

Other changes occurring during immunosenescence include reduction in cytokine signaling responses, increased baseline levels of phospho-STAT, and elevation in memory cell populations (Shen-Orr et al. (2016). Defective Signaling in the JAK-STAT Pathway Tracks with Chronic Inflammation and Cardiovascular Risk in Aging Humans, Cell Systems. 3(4):374-384.E4, which is incorporated by reference in its entirety for all purposes). Of notable clinical importance, immunosenescence also results in defects in antibody responses, with many older individuals failing to generate protective antibody titers following vaccination (Furman D et al. 2013). Apoptosis and other immune biomarkers predict influenza vaccine responsiveness. Molecular Systems Biology 9:659, which is incorporated by reference in its entirety for all purposes).

Immunosenescence impacts both the host's capacity to respond to infections and the development of long-term immune memory, especially by vaccination. Immunosenescence is associated with the accumulation of memory and effector cells as a result of repeated infections and by continuous exposure to antigens (inhalant allergens, food, etc.). This chronic inflammation characterizes immunosenescence and can have a significant impact on survival and fragility. Immunosenescence can also be associated with remodeling of the immune system caused by oxidative stress.

Immunosenescence can occur from an imbalance between inflammatory and anti-inflammatory mechanisms producing chronic inflammation. This chronic inflammation can be due to chronic antigen stimulation occurring over the course of life and to the oxidative stress that involves the production of oxygen free radicals and toxic products. These factors are able to modify the potential of apoptotic lymphocytes, and this remodeling of the lymphocyte compartment and the chronic expression of proinflammatory cytokines are implicated in the processes of longevity and diseases related to immunosenescence.

Canonical acute inflammation proteins (C-reactive protein, Interleukin-6, etc.) have been associated with immunosenescence in previous studies, but the relationship with systemic chronic inflammation has not yet been established. Using a well-known marker for immunosenescence (the frequency of naïve CD8 (+) T cells) contribution of iAge to immunosenescence was estimated after controlling for Age, CMV, and sex by a multiple regression model. Age was the strongest contributor to changes in naïve CD8 (+) T cells followed by iAge, CMV (negative contributors) and sex (frequency of total CD8 (+) T cells in females was 24% vs. 30% in males). iAge was significantly correlated with the frequency of naïve CD8 (+) T cells to a similar extent to CMV positivity. Chronological age was the strongest contributor (P<10⁻¹⁵), followed by iAge (P<10⁻⁵), CMV (P<10⁻³) and gender (P=0.012) (A).

The effect of chronic inflammation on the immune response was also measured using a functional immune assay (phospho-flow) in which cells are stimulated ex vivo and the phosphorylation of various intracellular proteins is measured by using antibodies against phosphorylated forms of these proteins. In particular, the responses to four independent stimuli (Interferon-alpha, Interleukin-6, Interleukin-10 and Interleukin-21) were measured in a total of 818 individuals and the fold-increase in phospho-STAT1, -STAT3 and -STAT5 in B cells, total CD4 (+) T cells (and the CD45RA(+) and CD45RA(−) subsets), total CD8 (+) T cells (and the CD45RA(+) and CD45RA(−) subsets), and in monocytes were determined. Multiple regression analysis controlling age, CMV and sex, surprisingly showed there was a general decrease of the B cell and T cell responses to stimuli and an overall potentiation of the monocyte responses associated with increasing iAge (combined P<10⁻⁵).

These results demonstrate that iAge is an important immune predictor of immune function decline (immunosenescence) and can be used as a ‘metric’ for immunological health.

Immunosenescence is associated with lowered ability of the immune system to kill cancer cells, protect against infections from pathogenic organisms, and produce efficacious response to vaccines. Treating a subject to lower their iAge can reduce the immunosenescence in the subject and improve the ability of the subject's immune system to kill cancer cells, protect against infections from pathogenic organisms, and produce efficacious responses to vaccines. Agents and methods for lowering iAge and thereby reducing immunosenescence are described below.

### Immunotherapies

In recent years, there has been a sharp rise in the development and implementation of cancer immunotherapies against cancer. The approval of anti-cytotoxic T-lymphocyte-associated protein 4 (CTLA-4) and anti-programmed cell death protein 1 (PD-1) antibodies has resulted in significant improvements in disease outcomes for a variety of cancers. Unlike chemo- and radiotherapy, which aim to interfere with tumor cell growth and survival, immunotherapies indirectly target the tumor by boosting the anti-tumor immune responses of the patient. Despite the huge success of these therapies in many forms of cancer, the success rates are generally low and biomarkers to define objective clinical responses are still lacking.

The biological foundation of current cancer immunotherapies is the concept of cancer immune surveillance, which proposes that the immune system eliminates tumor cells because these possess new antigens and trigger an immune reaction with regression of the tumor and no clinical signs of its existence. In a seminal study to test this theory, it was shown that chemically-induced sarcomas grew faster and more aggressively in immune-incompetent mice than in wild type animals because the former lack lymphocytes (have a engineered mutation of the recombination-activating (RAG) gene) or their inability to respond to IFNγ, either because of the loss of the IFNγ receptor gene, or the STAT-1 gene. Immune surveillance was shown by using mice with a double mutation RAG2^{-/-}/STAT1^{-/-} which spontaneously developed tumors. These tumors resemble some of the major malignancies of humans, such as breast, lung, or colon. Cancer immunoediting was shown by transplanting tumors between mice. When a tumor was transplanted from an immune incompetent mouse to a immune competent mouse, 40% of the tumors were rejected. Whereas no rejection occurred when transplants were performed using tumors from syngeneic immune-competent mice. This clearly demonstrated that immunoediting had occurred in immune-competent animals, even if they were incapable of rejecting their own tumor, enabling their escape from immune-surveillance. After decades of follow-up work a novel theory of tumor immunity was introduced. The theory proposed three steps: 1) elimination of tumors at early stages (immune surveillance hypothesis), 2) equilibrium which refers to the state in which the immune system controls the tumor, and 3) escape when tumor cells are immune-edited and grow without immune control. This three E's theory is still the theory accepted worldwide as the basis to understand the interaction of cancer cells with the immune system. The theory also paved the way for the exploding field of cancer immunotherapy.

Immunotherapy for cancer boosts the body's natural defenses to fight cancer. It uses substances made by the body or in a laboratory to improve or restore immune system function. Cancer immunotherapies include, for example, monoclonal antibodies, immune checkpoint inhibitors, cancer vaccines, immune cells modified with, for example, chimeric antigen receptors, and other nonspecific immunotherapies that boost the immune system function or action by, for example, specifically targeting cancer cells, overcoming inhibition of the immune system (e.g., by myeloid suppressor cells), etc.

Monoclonal antibodies for treating cancer include, for example, anti-CD20 antibody (e.g., Bexxar®, Zevalin®, Rituxan®, Gazyvaro®, Arzerra®), anti-Her2 antibody (e.g., Herceptin®, Kadcyla®, Perjeta®), anti-CD30 antibody (e.g., Adcetris®), anti-CD19 and anti-CD3 bispecific antibody (e.g., Blincyto®), anti-VegF antibody (e.g., Avastin®, Cyramza®), anti-EGFR antibody (e.g., Erbitux®, Portrazza®, Vectibix®), anti-PDGFR-α antibody (e.g., Lartruvo®), anti-CD38 antibody (e.g., Darzalex®), antiSLAMF7 antibody (e.g., Empliciti®), anti-GD2 antibody (e.g., Unituxin®), anti-CD19 antibody (e.g., Blincyto®), anti-RANKL antibody (e.g., Xgeva®, Prolia®), anti-EpCAM and anti-CD3 antibody (e.g., Removab®), anti-EpCAM antibody (e.g., Proxinium®), anti-CD52 antibody (e.g., Campath®), and anti-CD33 antibody (e.g., Mylotarg®).

Checkpoint inhibitors for treating cancer include, for example, Nivolumab (Opdivo), Pembrolizumab (Keytruda), Atezolizumab (Tecentriq), Ipilimumab (Yervoy), Durvalumab (Imfinzi®), Avelumab (Bavencio®), Lirilumab, and BMS-986016 (Relatlimab). Nivolumab, Atezolizumab, Pembrolizumab, Durvalumab, and Avelumab act at the checkpoint protein PD-1/PD-L1 and inhibit apoptosis of anti-tumor immune cells. Ipilimumab acts at CTLA4 and prevents CTLA4 from downregulating activated Tcells in the tumor. Lirilumab acts at KIR and facilitates activation of Natural Killer cells. BMS-986016 acts at LAG3 and activates antigen-specific T-lymphocytes and enhances cytotoxic T cell-mediated lysis of tumor cells.

Chimeric Antigen Receptors for treating cancer include, for example, an antiCD19 CAR in T-cells (e.g., Kymriah® and Yescarta®). CAR therapy can also be directed at a variety of tumor-associated antigens including, for example, 4-1BB, 5T4, adenocarcinoma antigen, alpha-fetoprotein, BAFF, B-lymphoma cell, C242 antigen, CA125, carbonic anhydrase 9 (CA-IX), C-MET, CCR4, CD152, CD19, CD20, CD21, CD22, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, alpha 5β1 lintegrin, integrin αvβ33, MORAb-009, MS4A1, MUC1, mucin CanAg, Nglycolylneuraminic acid, NPC-1C, PDGF-Rα, PDL192, phosphatidylserine, prostatic carcinoma cells, RANKL, RON, ROR1, SCH 900105, SDC1, SLAMF7, TAG-72, tenascin C, TGF β2, TGF-β., TRAIL-R1, TRAIL-R2, tumor antigen CTAA16.88, VEGFA, VEGFR-1, VEGFR2, 707-AP, ART-4, B7H4, BAGE, β-catenin/m, Bcr-abl, MN/C IX antibody, CAMEL, CAP-1, CASP-8, CD25, CDC27/m, CDK4/m, CT, Cyp-B, DAM, ErbB3, ELF2M, EMMPRIN, ETV6-AML1, G250, GAGE, GnT-V, Gp100, HAGE, HLA-A*0201-R170I, HPV-E7, HSP70-2M, HST-2, hTERT (or hTRT), iCE, IL-2R, IL5, KIAA0205, LAGE, LDLR/FUT, MAGE, MART-1/melan-A, MART-2/Ski, MC1R, myosin/m, MUM-1, MUM-2, MUM-3, NA88-A, PAP, proteinase-3, p190 minor bcr-abl, Pml/RARα, PRAIVIE, PSA, PSM, PSMA, RAGE, RU1 or RU2, SAGE, SART-1 or SART-3, survivin, TPI/m, TRP-1, TRP-2, TRP-2/INT2, WT1, NY-Eso-1 or NY-Eso-B or vimentin.

Cancer vaccines include, for example, human papilloma virus (HPV) vaccine, dendritic cell vaccines (e.g., Provenge® for prostate cancer), tumor cell vaccines, antigen vaccines, oncolytic virus vaccines (e.g., Imlygic™), Non-Hodgkin's lymphoma and mantle cell lymphoma vaccine (e.g., BioVaxID™), breast cancer vaccine (e.g., Neuvax™), brain cancer vaccine (e.g., DCVax™, CDX-110™), pancreatic cancer vaccine (e.g., GVAX Pancreas, HyperAcute™ Pancreas), colorectal cancer vaccine (e.g., Imprime PGG®), bladder cancer vaccine (e.g., BCG™), solid tumor vaccine (e.g., OK432™), lung cancer and gastrointestinal cancer vaccine (e.g., PSK™), cervical cancer vaccine (e.g., Schizophyllan™), and stomach cancer vaccine (e.g., Lentinan™)

Other immunotherapies for treating cancer include, for example, an IL-2 diphtheria toxin fusion protein (e.g., Ontak®),

### Types of Cancer

Cancers that can be treated with the methods described herein, include, for example, the approved indications for the FDA approved immunotherapies, such as melanoma, non-small cell lung cancer, Head and Neck squamous cell cancer, classical Hodgkin's lymphoma, primary mediastinal large B-cell lymphoma, urothelial carcinoma, microsatellite instability-high cancer, gastric cancer, cervical cancer, hepatocellular carcinoma, Merkel Cell carcinoma, renal cell carcinoma (Keytruda®); advanced or metastatic urothelial carcinoma, unresectable, stage III non-small cell lung cancer (Imfinzi®); unresectable or metastatic melanoma, metastatic non-small cell lung cancer, advanced renal cell carcinoma, classical Hodgkin's lymphoma, recurrent or metastatic squamous cell carcinoma, advanced or metastatic urothelial carcinoma, microsatellite instability high, or mismatch repair deficient metastatic colorectal cancer, hepatocellular carcinoma (Opdivo®); urothelial carcinoma, non-small cell lung cancer, triple negative breast cancer, small cell lung cancer (Tecentriq®); metastatic Merkel cell carcinoma (Bavencio®); unresectable of metastatic melanoma, advanced renal cell carcinoma, microsatellite instability high, or mismatch repair deficient metastatic colorectal cancer (Yervoy®); refractory diffuse B-cell lymphoma, relapsed or refractory acute lymphoblastic leukemia (Kymriah®); or diffuse large B-cell lymphoma, primary mediastinal B-cell lymphoma, High grade B-cell lymphoma (Yescarta®).

Cancers that can be treated with the methods described herein, also include, for example the indications under development such as, acute myeloid leukemia, bladder cancer, squamous cell carcinoma of the head and neck, chronic lymphocytic leukemia, multiple myeloma, metastatic solid malignancies (Lirilumab™); or melanoma, advanced colorectal cancer, advanced Chordoma, metastatic melanoma, gastro/esophageal cancer, solid tumors, gastric cancer, advanced renal cell cancer, advanced non-small cell lung cancer (Relatlimab™).

Other cancers that can be treated with the methods herein include, for example, sarcoma, carcinoma, melanoma, chordoma, malignant histiocytoma, mesothelioma, glioblastoma, neuroblastoma, medulloblastoma, malignant meningioma, malignant schwannoma, leukemia, lymphoma, myeloma, myelodysplastic syndrome, myeloproliferative disease. In some embodiments, the cancer is a leukemia, lymphoma, myeloma, myelodysplastic syndrome, and/or myeloproliferative disease.

### Vaccines

Vaccines can be substances used to stimulate a protective immune response in a subject (e.g., an antibody response or a cell mediated response) and provide immunity against one or several diseases. Vaccines protect against more than many debilitating or life-threatening diseases/infectious agents, including for example, adenovirus, anthrax, cervical cancer, chicken pox, cholera, dengue, diphtheria, Haemophilus influenza, hepatitis A, hepatitis B, hepatitis E, HPV, influenza, Japanese encephalitis, malaria, measles, meningitis, meningococcal (MenACWY), serogroup B meningococcal, mumps, pneumococcus, polio, rabies, rotavirus, rubella, shingles, small pox, tetanus, tuberculosis, typhoid, varicella, whooping cough, and yellow fever.

Vaccines can be prepared from the causative agent of a disease, its products, or a synthetic substitute, treated to act as an antigen without inducing the disease. Examples of vaccine types include, for example, live or attenuated vaccines (e.g., measles, mumps, rubella, varicella, influenza, coronavirus, rotavirus, zoster, yellow fever), inactivated or killed vaccines (e.g., polio, hepatitis A, rabies), toxoid (inactivated toxoid) vaccines (e.g., diphtheria, and tetanus), and subunit/conjugate vaccines (e.g., hepatitis B, influenza, coronavirus, Haemophilus influenza type b, pertussis, pneumococcal, meningococcal, HPV).

Attenuated vaccines can be made in several different ways. Some of the most common methods involve passing the disease-causing virus through a series of cell cultures or animal embryos (typically chick embryos). Using chick embryos as an example, the virus is grown in different embryos in a series. With each passage, the virus becomes better at replicating in chick cells, but loses its ability to replicate in human cells. A virus targeted for use in a vaccine may be grown through—“passaged” through—upwards of 200 different embryos or cell cultures. Eventually, the attenuated virus will be unable to replicate well (or at all) in human cells, and can be used in a vaccine. All of the methods that involve passing a virus through a non-human host produce a version of the virus that can still be recognized by the human immune system, but cannot replicate well in a human host. When the resulting vaccine virus is given to a human, it will be unable to replicate enough to cause illness, but will still provoke an immune response that can protect against future infection.

Killed or inactivated vaccines can be created by inactivating a pathogen, typically using heat or chemicals such as formaldehyde or formalin. This destroys the pathogen's ability to replicate, but keeps it “intact” so that the immune system can still recognize it. (“Inactivated” is generally used rather than “killed” to refer to viral vaccines of this type, as viruses are generally not considered to be alive.) Because killed or inactivated pathogens can't replicate at all, they can't revert to a more virulent form capable of causing disease (as discussed above with live, attenuated vaccines). However, these vaccines tend to provide a shorter length of protection than live vaccines, and are more likely to require boosters to create long-term immunity.

Immunizations created using inactivated toxins are called toxoids. Toxoids can actually be considered killed or inactivated vaccines, but are sometimes given their own category to highlight the fact that they contain an inactivated toxin, and not an inactivated form of bacteria.

Both subunit and conjugate vaccines contain only pieces of the pathogens they protect against. Subunit vaccines use only part of a target pathogen to provoke a response from the immune system. This may be done by isolating a specific protein from a pathogen and presenting it as an antigen on its own. The acellular pertussis vaccine and influenza vaccine (in shot form) are examples of subunit vaccines. Another type of subunit vaccine can be created via genetic engineering. Conjugate vaccines can be made using a combination of two different components. Conjugate vaccines, however, are made using pieces from the coats of bacteria. These coats are chemically linked to a carrier protein, and the combination is used as a vaccine.

### Pathogens

Pathogenic organisms are capable of causing disease in a subject. A human pathogen is capable of causing illness in humans. Common examples of pathogenic organisms include specific strains of bacteria such as, for example, Actinomyces israelii, Bacillus anthracis, Bacteroides fragilis, Bordetella pertussis, Borrelia, Brucella, Campylobacter jejuni, Chlamydophila psittaci, Corynebacterium diphtheria, Ehrlichia, Enterococcus, Francisella tularensis, Haemophilus influenza, Helicobacter pylori, Klebsiella pneumoniae, Legionella pneumophila, Leptospira species, Listeria monocytogenes, Mycobacterium, Mycoplasma pneumoniae, Pseudomonas aeruginosa, Nocardia asteroids, Rickettsia rickettsia, Salmonella, Shigella, Treponema pallidum, Vibrio cholera, Yersinia pestis, Listeria E. coli, Staphylococcus, Streptococcus, Neisseria, Clostridia, Chlamydia, mycoplasmas.

Pathogenic organisms also include viruses such as, for example, adenoviruses, herpesviruses, influenza, coronavirus, hepatitis, poxviruses, papovaviruses, paramyxoviruses, coronaviruses, picornaviruses, Reoviruses, togaviruses, flaviviruses, arenaviruses, rhabdoviruses, retroviruses, hepadnaviruses, Cryptosporidium.

Pathogenic organisms include fungi such as, for example, Candida, Aspergillus, Cryptococcus, Histoplasma, Pneumocystis, and Stachybotrys.

Pathogens also include the above organisms which are the target of vaccines.

Anti-pathogen therapies can include, for example, antibiotics for bacterial pathogens, anti-viral therapies for viral pathogens, and anti-fungal therapies for fungal pathogens. Antibodies can also be administered for the treatment of certain infectious diseases caused by bacteria, viruses or fungi.

Cancer Treatments Using iAge

Subjects with cancer who are candidates for immunotherapy (as described above) have their blood drawn and an iAge and CRS are calculated as described above. If the subject's iAge places them in the youngest iAge quartile for their age group (see Table 1) they can be classified as responders and move forward with the immunotherapy. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile.

Alternatively, if the subject's iAge places them in the youngest iAge quintile for their age group (see Table 1) they can be classified as responders and move forward with the immunotherapy. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile.

Still alternatively, if the subject's iAge places them in the youngest iAge tertile for their age group (see Table 1) they can be classified as responders and move forward with the immunotherapy. If the subject's iAge places them in the middle tertile, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile.

Subjects who are candidates for vaccinations (e.g., the elderly) can have their blood drawn and an iAge and CRS are calculated as described above. If the subject's iAge places them in the youngest iAge quartile for their age group (see Table 1) they can be classified as responders and move forward with the vaccination. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Alternatively, if the subject's iAge places them in the youngest iAge quintile for their age group (see Table 1) they can be classified as responders and move forward with the vaccination. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Still alternatively, if the subject's iAge places them in the youngest iAge tertile for their age group (see Table 1) they can be classified as responders and move forward with the vaccination. If the subject's iAge places them in the middle tertile, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Subjects who have been exposed to a pathogenic organism, are infected with a pathogenic organism, and/or are susceptible to infection by a pathogenic organism can have their blood drawn and an iAge and CRS are calculated as described above. If the subject's iAge places them in the youngest iAge quartile for their age group (see Table 1) they can be classified as responders and move forward with standard treatment for the pathogenic organism. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can move forward with standard treatment for the pathogenic organism. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile. Subjects classified as nonresponders can also be treated with more aggressive therapies and/or higher doses of therapeutics to account for the immunosenescence in the subject.

Alternatively, if the subject's iAge places them in the youngest iAge quintile for their age group (see Table 1) they can be classified as responders and move forward with standard treatment for the pathogenic organism. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can move forward with standard treatment for the pathogenic organism. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile. Subjects classified as nonresponders can also be treated with more aggressive therapies and/or higher doses of therapeutics to account for the immunosenescence in the subject.

Still alternatively, if the subject's iAge places them in the youngest iAge tertile for their age group (see Table 1) they can be classified as responders and move forward with standard treatment for the pathogenic organism. If the subject's iAge places them in the middle tertile, the subject's blood cells (e.g., CD4+ and CD8+ cells) are stimulated and Jak-STAT activity is measured (see, e.g., Example 1 below). Subject's whose JakSTAT activity places them in the highest quartile can be classified as responders and can move forward with standard treatment for the pathogenic organism. Subjects whose JakSTAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile. Subjects classified as nonresponders can also be treated with more aggressive therapies and/or higher doses of therapeutics to account for the immunosenescence in the subject.

### Cardiovascular Disease

Cardiovascular disease include a class of diseases that involve the heart, the blood vessels (arteries, capillaries, and veins) or both. Cardiovascular disease refers to any disease that affects the cardiovascular system, principally cardiac disease including cardiomyopathies, vascular diseases of the brain and kidney, and peripheral arterial disease. Cardiovascular disease can refer to a disease that primarily affects the heart, and can be referred to as cardiac disease. Cardiovascular disease can refer to a disease in which the pathology begins with cardiac damage, malfunction, or malformation, as opposed to disease in which cardiac damage, malfunction, or malformation is a result of a primary pathology present at a site remote from the heart (e.g., cardiovascular disease as a comorbidity to another disease or condition). For example, heart failure, cardiac dysrhythmias (abnormalities of heart rhythm including increased QT duration and atrial flutter and/or fibrillation), inflammatory heart disease including endocarditis (inflammation of the inner layer of the heart, the endocardium, most commonly the heart valves); inflammatory cardiomegaly (enlarged heart, cardiac hypertrophy); myocarditis (inflammation of the myocardium); valvular heart disease; congenital heart disease; and rheumatic heart disease (heart muscle and valve damage due to rheumatic fever caused by streptococcal bacteria infections) are examples of cardiac damage, malfunction, or malformation in which the primary pathology can be or is present in the heart, and subsequently can result in vascular or other systemic disease. Alternatively, coronary heart disease (also ischaemic heart disease or coronary artery disease); hypertensive heart disease (diseases of the heart secondary to high blood pressure); cor pulmonale (failure at the right side of the heart with respiratory system involvement); cerebrovascular disease (disease of blood vessels that supplies to the brain such as stroke); peripheral arterial disease (disease of blood vessels that supplies to the arms and legs); and atherosclerosis are a result of pathology present initially at a site remote from the heart. Cardiovascular disease initiated either at the heart or at a site remote from the heart can result in heart failure. Cardiovascular disease can include disease in which the initial pathology is at a site remote from the heart. Cardiovascular disease also includes conditions affecting the heart, heart valves, and vasculature (e.g., arteries and veins) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

Cardiomyopathy includes one or more conditions (e.g., 1, 2, 3, 4, 5, 6, 7, 8, 9, 10, or more of) selected from the group consisting of increased QT duration, arrhythmias, myocardial ischemia, hypertension and thromboembolic complications, myocardial dysfunction, cardiomyopathy, heart failure, atrial fibrillation, cardiomyopathy and heart failure, heart failure and LV dysfunction, atrial flutter and fibrillation, and, heart valve damage and heart failure. In certain embodiments, cardiomyopathy does not include cardiomyopathy as a comorbidity to another disease or condition.

Heart failure often called congestive heart failure (CHF) or congestive cardiac failure (CCF), includes conditions that occur when the heart is unable to provide sufficient pump action to maintain blood flow to meet the needs of the body. Heart failure can cause a number of symptoms including shortness of breath, leg swelling, and exercise intolerance. The condition is typically diagnosed by patient physical examination and confirmed with echocardiography. Common causes of heart failure include myocardial infarction and other forms of ischemic heart disease, hypertension, valvular heart disease, and cardiomyopathy.

Cardiovascular disease includes atherosclerosis a chronic disease process characterized by lipid deposits and fibrosis of the intima, irregularly distributed in large and medium sized arteries. The disease is progressive and most often becomes clinically manifest in the middle-aged and elderly. When severe, the atherosclerotic plaque causes a reduction of the cross-sectional area of the arterial lumen, with and without thrombosis. Atherosclerotic plaques can occur in essentially any or all of the blood vessels of the body, resulting in cardiovascular diseases involving the heart (e.g., acute coronary syndrome, heart failure, and myocardial infarction), the brain (e.g., stroke, transient ischemic attack, and brain infarction), the kidney (e.g., acute and chronic kidney disease, hypertension), and the extremities (e.g., peripheral vascular disease, lower and/or upper extremity claudication, and lower and/or upper extremity ischemia). Resultant ischemic manifestations include: angina pectoris, rayocardial infarction, stroke, intermittent claudication, gangrene of the lower extremities, and renovascular hypertension. Atherosclerosis can be considered to be an inflammatory disease. For example, the lesions of atherosclerosis appear to represent a series of highly-specific cellular and molecular responses that can be described as an inflammatory disease. See, e.g., Ross, “Atherosclerosis—An inflammatory disease” N Engl J Med (1999), 340:115-126; the publications cited in Ross (1999); and subsequent publications that cite Ross (1999); each of which is incorporated herein in reference in its entirety.

A subject can be identified as having cardiovascular disease by the presence of any one of: documented coronary artery disease, documented cerebrovascular disease, documented carotid disease, documented peripheral arterial disease, or combinations thereof. A subject can also be identified as having cardiovascular disease if the subject is at least 45 years old and: (a) has one or more stenosis of greater than 50% in two major epicardial coronary arteries; (b) has had a documented prior MI; (c) has been hospitalized for high-risk NSTE ACS with objective evidence of ischemia (e.g., ST-segment deviation and/or biomarker positivity); (d) has a documented prior ischemic stroke; (e) has symptomatic artery disease with at least 50% carotid arterial stenosis; (0 has asymptomatic carotid artery disease with at least 70% carotid arterial stenosis per angiography or duplex ultrasound; (g) has an ankle-brachial index (“ABI”) of less than 0.9 with symptoms of intermittent claudication; and/or (h) has a history of aorto-iliac or peripheral arterial intervention (catheter-based or surgical).

Cardiovascular Treatments Using iAge and Cardiac Markers

Subjects with cardiovascular disease or at risk for cardiovascular disease have their blood drawn and an iAge, CRS, cardiac marker levels (MIG, LIF, SIRT3), and cAge are calculated as described above. If the subject's iAge, CRS, cardiac marker levels (MIG, LIF, SIRT3), and/or cAge places them in the youngest quartile for their age group they can be classified as low risk for cardiovascular disease and move forward with the standard therapy (CVD patients) or no therapy (patients at risk but no CVD at the time). If the subject's iAge, CRS, cardiac marker levels (MIG, LIF, SIRT3), and/or cAge places them in the middle two quartiles, the subject's blood cells (e.g., CD4+ and CD8+ cells) can be tested for Jak-STAT activity (see, e.g., Example 1 below). Subject's whose Jak-STAT activity places them in the highest quartile can be classified as low risk and move forward with standard therapy (CVD patients) or no therapy (patients at risk but no CVD at the time). Subjects whose Jak-STAT activity places them in the lower three quartiles can be classified as higher risk for cardiovascular disease and can be treated to lower iAge , CRS, cardiac marker levels (MIG, LIF, SIRT3), and/or cAge (and increase their Jak-STAT score) into a low risk group. If the subject's iAge, CRS, cardiac marker levels (MIG, LIF, SIRT3), and/or cAge places them in the oldest quartile, they can be classified as higher risk patients and can be treated to lower their iAge, CRS, cardiac marker levels (MIG, LIF, SIRT3), and/or cAge (see above) into a low risk group.

Alternatively, if the subject's iAge, CRS, cardiac marker levels (MIG, LIF, SIRT3), and/or cAge places them in the youngest iAge, CRS, cardiac marker levels (MIG, LIF, SIRT3), and/or cAge quintile for their age group (see Table 1) they can be classified as low risk and move forward with the standard therapy (CVD patients) or no therapy (patients with no CVD at the time). If the subject's iAge, CRS, cardiac marker levels (MIG, LIF, SIRT3), and/or cAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as low risk and move forward with the standard therapy (CVD patients) or no therapy (patients at risk but no CVD at the time). Subjects whose Jak-STAT activity places them in the lower three quartiles can be classified as higher risk and can be treated to lower iAge, CRS, cardiac marker levels (lower MIG, raise LIF, raise SIRT3), and/or cAge (and increase their Jak-STAT score) into a low risk group. If the subject's iAge places them in the oldest quintile, they can be classified as higher risk and can be treated to lower their iAge, CRS, cardiac marker levels (lower MIG, increase LIF, increase SIRT3), and/or cAge (see above) into a low risk group of a younger iAge quintile.

Still alternatively, if the subject's iAge, CRS, cardiac marker levels (MIG, LIF, SIRT3), and/or cAge places them in the youngest iAge tertile for their age group (see Table 1) they can be classified as low risk and move forward with the standard therapy (CVD patients) or no therapy (patients at risk but no CVD at the time). If the subject's iAge, CRS, cardiac marker levels (MIG, LIF, SIRT3), and/or cAge places them in the middle tertile, the subject's blood cells (e.g., CD4+ and CD8+ cells) are stimulated and Jak-STAT activity is measured (see, e.g., Example 1 below). Subject's whose Jak-STAT activity places them in the highest quartile can be classified as low risk and move forward with the standard therapy (CVD patients) or no therapy (patients at risk but no CVD at the time). Subjects whose Jak-STAT activity places them in the lower three quartiles can be classified as higher risk and can be treated to lower iAge, CRS, cardiac marker levels (lower MIG, increase LIF, increase SIRT3), and/or cAge (and increase their Jak-STAT score) into a low risk group. If the subject's iAge, CRS, cardiac marker levels (MIG, LIF, SIRT3), and/or cAge places them in the oldest tertile, they can be classified as higher risk and are treated to lower their iAge, CRS, cardiac marker levels (lower MIG, increase LIF, increase SIRT3), and/or cAge (see above) into a low risk group of a younger iAge, CRS, cardiac marker levels (MIG, LIF, SIRT3), and/or cAge tertile.

Agents for Improving iAge and Cardiac Markers

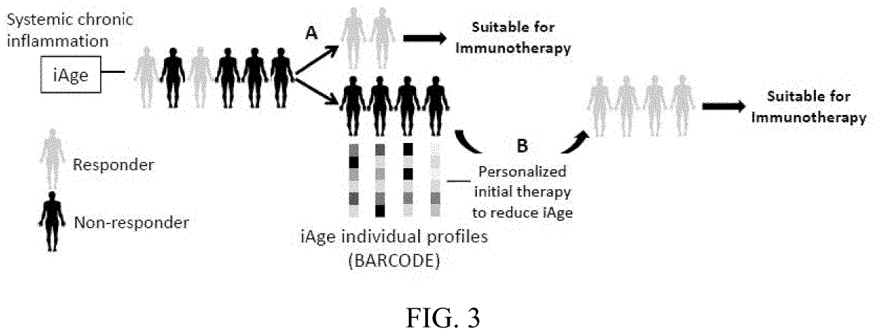

In addition to using iAge to classify patients (FIG. 3) or cAge to classify patients, these can be used to derive individual inflammatory profiles by comparing subject's individual protein levels with those of a population (e.g., of similar chronological age). The resulting signatures (or barcodes) are used for protein-compound association (PCI) analysis using the drugbank database (www.drugbank.ca) and a personalized initial therapy to reduce iAge can be generated (FIG. 3). Patients following personalized recommendations can be monitored weekly for changes in iAge and/or cAge until they reach optimal levels (below group average for a given age bracket) and they convert into a responder treatment phenotype (FIG. 3) and/or lower risk for cardiovascular disease. The patient is then classified as a responder and is suitable for immunotherapy treatment, or lower risk for CVD.

A subject may reduce their iAge with treatments that lower the levels of TRAIL, IFNG, GROA, IL2, TGFA, PAI1, and/or LIF to their optimal levels for a person's chronological age. A subject may reduce their cAge with treatments that raise the levels of LIF or SIRT3 A subject may also reduce their iAge with treatments that raise the levels of MIG, EOTAXIN, LEPTIN, IL-1B, or MIP1A to their optimal levels for a person's age. A subject may also reduce their cAge with treatments that lower the levels of MIG.

A subject may also reduce their iAge and/or cAge by reducing any systemic chronic inflammation, using any of the following, whether alone or in combination: (1) pharmacological treatment, including without limitation anti-inflammatory drugs (NSAIDs such as, for example, aspirin, ibuprofen, naproxen, diclofenac, celecoxib, oxaprozin, piroxicam, indomethacin, meloxicam, fenoprofen, diflunisal, etodolac, ketorolac, meclofenamate, nabumetone) or corticosteroids (e.g., glucocorticoids, mineralocorticoids); (2) neutraceuticals or nutritional supplements, including without limitation fish oil, lipoic acid, and curcumin, or spices/herbs such as ginger, garlic, turmeric, hyssop, cannabis, Harpagophytum procumbens, and cayenne; (3) dietary change, including without limitation increasing the intake of foods that are high in antioxidants and polyphenols, such as olive oil, leafy greens (e.g., kale and spinach), broccoli, avocados, green tea, bell peppers, chili peppers, mushrooms, dark chocolate, cocoa, tomatoes, fatty fish (e.g., salmon, sardines, herring, anchovies, and mackerel), nuts (walnuts and almonds), and fruits (e.g., cherries, blackberries, blueberries, raspberries, strawberries, grapes, and oranges), and/or decreasing the intake of foods that can increase inflammation such as refined carbohydrates (e.g., white bread and pastries), high-fructose corn syrup, refined sugar, processed and packaged food, fried foods, red meat, excessive alcohol, and processed meat; and (4) lifestyle changes including without limitation eliminating or reducing smoking and alcohol intake, maintaining a healthy body weight, and reducing stress levels.

Cardiac markers can be improved by providing a subject with treatments that improve the level of the cardiac marker (lowering cAge). A subject's cardiac marker score (cAge) can be lowered by reducing the MIG in a patient, increasing Sirtuin-3 in the patient, increasing LIF in the patient, and/or reducing cell signaling from CXCR3 (the receptor for MIG). A variety of agents are known which can reduce MIG expression, increase Sirtuin-3 expression, increase LIF activity (or LIF like activity), and/or act as antagonists for CXCR3 (the receptor for MIG).

Agents that can lower MIG (and so improve the cardiac marker and iAge marker) include, for example arsenic trioxide, Roxarsone, Selenium, and/or a variety of antibodies. Antibodies include, for example, MIG-2F5.5 (anti-human CXCL9 antibody, BioLegend Cat. #740072), Anti-human CXCL9 antibody, NSJ Bioreagents, Cat #R30501, Mouse MAb clone 49106 (anti-human CXCL9, R&D Systems Cat #MAB392), Mouse monoclonal MAb for human CXCL9 (neutralizing, GeneTex, Cat #GTX52673), Mouse monoclonal anti-human CXCL9 antibody (OriGene, Cat#PM1209P), MIG Antibody (MM0220-7F11) (Novus Biologicals, NBP2-12236), MIG Antibody (1F5) (Novus Biologicals, H00004283-M06), Mouse MAb anti human CXCL9 (ThermoFisherCat #MA5-23746, Cat #MA5-30320, Cat #MA5-23628, Cat #MA5-23544).

Arsenic trioxide (As₂O₃), a component of traditional Chinese medicine, has been used successfully for the treatment of acute promyelocytic leukemia (APL), and As₂O₃ is of potential therapeutic value for the treatment of other promyelocytic malignancies and some solid tumors including breast cancer. As₂O₃ treatment changed the expression level of several genes that involved in cell cycle regulation, signal transduction, and apoptosis. Notably, As₂O₃ treatment increased the mRNA and protein levels of the cell cycle inhibitory proteins, p21 and p27. Interestingly, knocking down p21 or p27 individually did not alter As₂O₃-induced apoptosis and cell cycle arrest; however, the simultaneous down-regulation of both p21 and p27 resulted in attenuating of G1, G2/M arrest and reduction in apoptosis, thus indicating that p21 and p27 as the primary molecular targets of As₂O₃.

Roxarsone is an organoarsonic acid where the organyl group is 4-hydroxy-3-nitrophenyl. It has a role as a coccidiostat, an antibacterial drug, an agrochemical and an animal growth promotant. It is an organoarsonic acid and a member of 2-nitrophenols. Roxarsone was found to exhibit a higher angiogenic index than As^{III} at lower concentrations. Increased endothelial nitric oxide synthase (eNOS) activity was observed for roxarsone but not for As^{III}-induced angiogenesis. However, As^{III} caused more rapid and pronounced phosphorylation of eNOS.

Selenium (Se) is a potential anticarcinogenic nutrient, and the essential role of Se in cell growth is well recognized but certain cancer cells appear to have acquired a survival advantage under conditions of Se-deficiency. Se can exert its effects through increasing the expression of a humoral defense gene (A2M) and tumor suppressor-related genes (IGFBP3, HHIP) while decreasing pro-inflammatory gene (MIG, HSPB2) expression.

Agents that raise Sirtuin-3 levels include, for example, Berberine and Resveratrol. Berberine (molecular formula C₂₀H₁₉NO₅ and molecular weight of 353.36) is the main active component of an ancient Chinese herb Coptis chinensis French, which has been used to treat diabetes for thousands of years. Berberine is an Over-the-Counter (OTC) drug, which is used to treat gastrointestinal infections in China. Berberine has been shown to regulate glucose and lipid metabolism in vitro and in vivo. Berberine is also a potent oral hypoglycemic agent with beneficial effects on lipid metabolism.

Resveratrol (3,5,4′-trihydroxy-trans-stilbene) belongs to polyphenols' stilbenoids group, possessing two phenol rings linked to each other by an ethylene bridge. This natural polyphenol has been detected in more than 70 plant species, especially in grapes' skin and seeds, and was found in discrete amounts in red wines and various human foods. It is a phytoalexin that acts against pathogens, including bacteria and fungi. As a natural food ingredient, numerous studies have demonstrated that resveratrol possesses a very high antioxidant potential. Resveratrol also exhibit antitumor activity, and is considered a potential candidate for prevention and treatment of several types of cancer. Indeed, resveratrol anticancer properties have been confirmed by many in vitro and in vivo studies, which shows that resveratrol is able to inhibit all carcinogenesis stages (e.g., initiation, promotion and progression). Even more, other bioactive effects, namely as anti-inflammatory, anticarcinogenic, cardioprotective, vasorelaxant, phytoestrogenic and neuroprotective have also been reported.

Agents that raise LIF levels include Aminodarone, arsenic trioxide, Azathioprine, Estradiol, Chlorambucil, Clomiphene, Coumaphos, Cyclosporine, decitabine, Cisplatin, Vincristine, Formaldehyde, Glucose, Hydrogen Peroxide, letrozole, Lindane, Methotrexate, Quercetin, Oxyquinoline, resorcinol, resveratrol, Silicon Dioxide, Tretinoin, and troglitazone.

The expression of LIF is regulated by many cytokines. In normal human bone marrow stromal cells, IL-1α, IL-1β, TGF-β and tumor necrosis factor-α (TNF-α) can all increase the transcription of LIF mRNA. The induction of LIF by IL-1β and TNF-α was also observed in gingival fibroblasts and several cell types in human airways. In addition, the induction of LIF expression by other cytokines, including IL-6, IL-2 has been observed in different cell types, including airway smooth-muscles and MT-2 cells. The expression of LIF can also be inhibited by some factors, including 1α, 25-dihydroxyvitamin D3 and dexamethasone. The analysis of the LIF promoter revealed that transcription factor STAT5 can bind to the LIF promoter and induce its expression in myeloid cell lines. In addition, the LIF promoter region contains several ETS binding sites. The binding of ETS transcription factors to the LIF promoter is critical for the induction of LIF in response to T cell activators.

Amiodarone is a primarily a class III antiarrhythmic and is one of the most commonly used anti-arrhythmic drugs. While the United States FDA has labeled amiodarone for the treatment of life-threatening ventricular arrhythmias, the drug is commonly used off-label to treat supraventricular tachyarrhythmias such as atrial fibrillation as well as for the prevention of ventricular tachyarrhythmias (VTs) in high-risk patients. Like other antiarrhythmic drugs of this class, amiodarone works primarily by blocking potassium rectifier currents that are responsible for repolarization of the heart during phase 3 of the cardiac action potential. This potassium channel-blocking effect results in increased action potential duration and a prolonged effective refractory period in cardiac myocytes. Unlike other class III agents, amiodarone also interferes with beta-adrenergic receptors, calcium channels, and sodium channels.

Clomiphene is an ovulatory stimulant designated chemically as 2-[p-(2-chloro-1,2-diphenylvinyl)phenoxy]triethylamine citrate (1:1). It has the molecular formula of C26H28ClNO.C6H8O7 and a molecular weight of 598.09. Clomiphene is capable of interacting with estrogen-receptor-containing tissues, including the hypothalamus, pituitary, ovary, endometrium, vagina, and cervix. It may compete with estrogen for estrogen-receptor-binding sites and may delay replenishment of intracellular estrogen receptors. Clomiphene initiates a series of endocrine events culminating in a preovulatory gonadotropin surge and subsequent follicular rupture. The first endocrine event in response to a course of clomiphene therapy is an increase in the release of pituitary gonadotropins.

Coumaphos is an organothiophosphate insecticide, an organic thiophosphate and an organochlorine compound. It has a role as an agrochemical, an acaricide, an antinematodal drug, an avicide and an EC 3.1.1.8 (cholinesterase) inhibitor. Coumaphos is used for control of a wide variety of insects on cattle and parasitic mites (Varroa jacobson) on bees. It is also used in veterinary medicine for the treatment of screwworms, maggots, and ear ticks on livestock. In humans coumaphos causes muscarinic effects (parasympathetic), nicotinic effects (sympathetic and motor), and CNS effects associated with massive overstimulation of the chlorinergic system.

Lindane also known as gamma-hexachlorocyclohexane (γ-HCH), gammaxene, and Gammallin is an organochlorine chemical and an isomer of hexachlorocyclohexane that has been used both as an agricultural insecticide and as a pharmaceutical treatment for lice and scabies. Lindane is a neurotoxin that interferes with GABA neurotransmitter function by interacting with the GABA_{A} receptor-chloride channel complex at the picrotoxin binding site. In humans, lindane affects the nervous system, liver, and kidneys, and may well be a carcinogen.

Oxyquinoline is a heterocyclic phenol and Oxyquinoline Sulfate is its salt, both of which are described as cosmetic biocides for use in cosmetic formulations. Oxyquinoline can be used as an antiseptic, disinfectant, and has pesticide properties. Oxyquinoline is also a chelating agent which has been used for the quantitative determination of metal ions.

Decitabine (5-aza-2′-deoxycytidine or 5-Aza-Cdr) is a cytosine analogue that was first synthesized in the early 1960s by Pliml and Sorm and is currently marketed as Dacogen® by Eisai (Tokyo, Japan). It differs from deoxycytidine by the substitution of nitrogen for carbon at the 5-position of the pyrimidine ring. It was noted to have an antileukemic effect in cell lines, with more potency in vitro than cytarabine. Initially, its cytotoxicity was attributed to its ability to impair DNA synthesis and cause DNA damage similar to other antimetabolites. At low doses, decitabine induces differentiation by reversing DNA methylation-induced gene silencing. Once inside a cell, decitabine is phosphorylated and activated by the enzyme deoxycytidine kinase to its triphosphate form aza-dCTP. It then competes with and replaces cytosine in the CpG (cytosine-guanosine dinucleotide) islands that occur in clusters in promoter regions. During subsequent cell divisions, aza-dCTP inhibits methylation of the promoter by forming a covalent bond with the enzyme DNA methyltransferase (DNMT), and thereby traps and contributes to degradation of the enzyme.

Chlorambucil and Cisplatin are alkylating agents used to treat cancer. Chlorambucil is in the class of nitrogen mustards, and Cisplatin is a platinum based-agent. Chlorambucil produces its anti-cancer effects by interfering with DNA replication and damaging the DNA in a cell. The DNA damage induces cell cycle arrest and cellular apoptosis via the accumulation of cytosolic p53 and subsequent activation of Bcl-2-associated X protein, an apoptosis promoter. Cisplatin crosslinks DNA in several different ways, interfering with cell division by mitosis. The damaged DNA elicits DNA repair mechanisms and activates apoptosis.

Vincristine is a chemotherapy drug that belongs to a group of drugs called vinca alkaloids. Vincristine works by stopping the cancer cells from separating into 2 new cells. Vincristine works partly by binding to the tubulin protein, stopping the tubulin dimers from polymerizing to form microtubules, causing the cell to be unable to separate its chromosomes during the metaphase. The cell then undergoes apoptosis.

Letrozole is an aromatase inhibitor which is used in the treatment of hormonally-responsive breast cancer after surgery. Letrozole is also for ovulation induction. Letrozole blocks the production of estrogens in this way by competitive, reversible binding to the heme of its cytochrome P450 unit. Letrozole has shown to reduce estrogen levels by 98 percent while raising testosterone levels.

Tretinoin is a derivative of vitamin A. It is used on the skin (topically) in the treatment of mild to moderate acne and on skin that has been damaged by excessive exposure to the sun. Tretinoin irritates the skin and causes the cells of the skin to grow (divide) and die more rapidly, increasing the turnover of cells. Tretinoin can also induce acute promyelocytic leukemia cells to differentiate and stops them from proliferating; in people there is evidence that it forces the primary cancerous promyelocytes to differentiate into their final form.

Estradiol is the main circulating oestrogen in women and reaches a plasma concentration of 30-400 pg/mL before menopause. Estradiol regulates growth and the development of the reproductive system, also, helps to maintain the osseous tissue, the central nervous system and the vasodilatation in the vascular tissue. The protective effect of Estradiol in the vasculature and against cardiovascular disease (CVD) has been demonstrated in several hormone replacement studies. Estradiol activates BK channels via a process that requires the presence of the β1 subunit. Valverde et al. were the first to propose that Estradiol affected BK channels by binding to β1, but it is still a matter of debate whether the agonistic action of Estradiol on BK channels is caused by its binding to the β1 subunit or to the α/β1 complex. Moreover, the molecular nature of the Estradiol binding site and the mode of action of the hormone are at present unknown. Acute application of Estradiol (100 nM) decreases smooth muscle excitability by activating BK channels. Notably, Estradiol or its membrane-impermeant form (E2-BSA) can induce a fast increase in BK channel activity in MCF-7 breast epithelial cancer cells with an EC50 of 80 pM reaching a maximal effect at 10 nM34. Rapid effects of Estradiol have also been reported in neurons of the area postrema where nanomolar concentrations of E2 can decrease the firing rate most probably by increasing BK current 35. All these examples underscore the physiological importance of the regulation of BK channels by E2 and made worthwhile efforts in determining the molecular nature of the interaction between this hormone and the BK channel.

Cyclosporine has been a core component of immunosuppression in both immune dysregulatory disorders and organ transplantation. For immune disorders involving ophthalmologic, dermatologic, hematologic, gastroenterologic, neurologic, or musculoskeletal systems, cyclosporine has demonstrated marked efficacy in relieving clinical symptoms and reversing pathological developments. Additionally, after the drug's implementation in transplantation medicine, rates of acute rejection and one-year graft survival have improved dramatically.

Methotrexate is a chemotherapy agent and immune system suppressant. It is used to treat cancer, autoimmune diseases, ectopic pregnancy, and for medical abortions. Types of cancers it is used for include breast cancer, leukemia, lung cancer, lymphoma, and osteosarcoma. Types of autoimmune diseases it is used for include psoriasis, rheumatoid arthritis, and Crohn's disease. It can be given by mouth or by injection. Methotrexate is an antimetabolite of the antifolate type. It is thought to affect cancer and rheumatoid arthritis by two different pathways. For cancer, methotrexate competitively inhibits dihydrofolate reductase (DHFR), an enzyme that participates in the tetrahydrofolate synthesis. The affinity of methotrexate for DHFR is about 1000-fold that of folate. DHFR catalyses the conversion of dihydrofolate to the active tetrahydrofolate. Folic acid is needed for the de novo synthesis of the nucleoside thymidine, required for DNA synthesis. Also, folate is essential for purine and pyrimidine base biosynthesis, so synthesis will be inhibited. Methotrexate, therefore, inhibits the synthesis of DNA, RNA, thymidylates, and proteins. For the treatment of rheumatoid arthritis (immune suppression), multiple mechanisms appear to be involved, including the inhibition of enzymes involved in purine metabolism, leading to accumulation of adenosine; inhibition of T cell activation and suppression of intercellular adhesion molecule expression by T cells; selective down-regulation of B cells; increasing CD95 sensitivity of activated T cells; and inhibition of methyltransferase activity, leading to deactivation of enzyme activity relevant to immune system function. Another mechanism of MTX is the inhibition of the binding of interleukin 1-beta to its cell surface receptor.

Troglitazone is an antidiabetic and anti-inflammatory drug, and a member of the drug class of the thiazolidinediones. Troglitazone is an oral antihyperglycemic agent which acts primarily by decreasing insulin resistance. Troglitazone is used in the management of type II diabetes. Troglitazone binds to nuclear receptors (PPAR) that regulate the transcription of a number of insulin responsive genes critical for the control of glucose and lipid metabolism. Troglitazone decrease nuclear factor kappa-B (NF-κB) and increase its inhibitor (IκB).

Azathioprine is a purine analogue with cytotoxic and immunosuppressive activity. Azathioprine is a prodrug that is converted by hepatic xanthine oxidase to its active metabolite 6-mercaptopurine (6-MP). 6-MP is further metabolized by hypoxanthine-guanine phosphoribosyltransferase (HGPRT) into 6-thioguanosine-5′-phosphate (6-thio-GMP) and 6-thioinosine monophosphate (6-thio-IMP), both inhibit nucleotide conversions and de novo purine synthesis. This leads to inhibition of DNA, RNA, and protein synthesis. As a result, cell proliferation may be inhibited, particularly in lymphocytes and leukocytes. Azathioprine an immunosuppressive agent in organ transplantation to prevent rejection and in autoimmune diseases as a corticosteroid sparing agent.

Quercetin, a flavonoid found in fruits and vegetables, has unique biological properties that may improve mental/physical performance and reduce infection risk. These properties form the basis for potential benefits to overall health and disease resistance, including anti-carcinogenic, anti-inflammatory, antiviral, antioxidant, and psychostimulant activities, as well as the ability to inhibit lipid peroxidation, platelet aggregation and capillary permeability, and to stimulate mitochondrial biogenesis. Quercetin is a naturally occurring polar auxin transport inhibitor. Quercetin inhibits lipopolysaccharide (LPS)-induced tumor necrosis factor α (TNF-α) production in macrophages and LPS-induced IL-8 production in lung A549 cells. Moreover, in glial cells it was even shown that quercetin can inhibit LPS-induced mRNA levels of TNF-α and interleukin IL-1α, this effect of quercetin resulted in a diminished apoptotic neuronal cell death induced by microglial activation. Quercetin inhibits production of inflammation-producing enzymes (cyclooxygenase (COX) and lipoxygenase (LOX)). It limits LPS-induced inflammation via inhibition of Src- and Syk-mediated phosphatidylinositol-3-Kinase (PI3K)-(p85) tyrosine phosphorylation and subsequent Toll Like Receptor 4 (TLR4)/MyD88/PI3K complex formation that limits activation of downstream signaling pathways in RAW 264.7 cells. It can also inhibit FcεRI-mediated release of pro-inflammatory cytokines, tryptase and histamine from human umbilical cord blood-derived cultured mast cells (hCBMCs); this inhibition appears to involve in inhibition of calcium influx, as well as phospho-protein kinase C (PKC).

Resorcinol is an organic compound with the formula C₆H₄(OH)₂. Resorcinol is used as an antiseptic and disinfectant in topical pharmaceutical products in the treatment of skin disorders and infections such as acne, seborrheic dermatitis, eczema, psoriasis, corns, calluses, and warts. It is also used to treat corns, calluses, and warts. It exerts a keratolytic activity.

Agents that reduce expression of CXCR3 (the receptor for MIG) include, for example, formaldehyde and taurine. Agents that are antagonists for CXCR3 include, for example, piperazinyl-piperidines (e.g., SCH546738), 8-azaquinazolinones (e.g., AMG 487), 3-phenyl-3H-quinazolin-4-ones, aryl piperazine, 4-aryl-5-piperazinylthiazoles, arylpiperazines, benzetimide derivatives, imidazolidines, imidazolium, lysergic acid derivative, diaminocyclobutenediones, zinc phthalocyanine, and NBI-74330. (See Andrews et al., J. Med. Chem. 59:2894-917 (2016), which is incorporated by reference in its entirety for all purposes). Chemical structures for specific antagonists of CXCR3 are found in Andrews 2016, and are hereby incorporated by reference in their entirety for all purposes. A few of the specific structures are shown below:

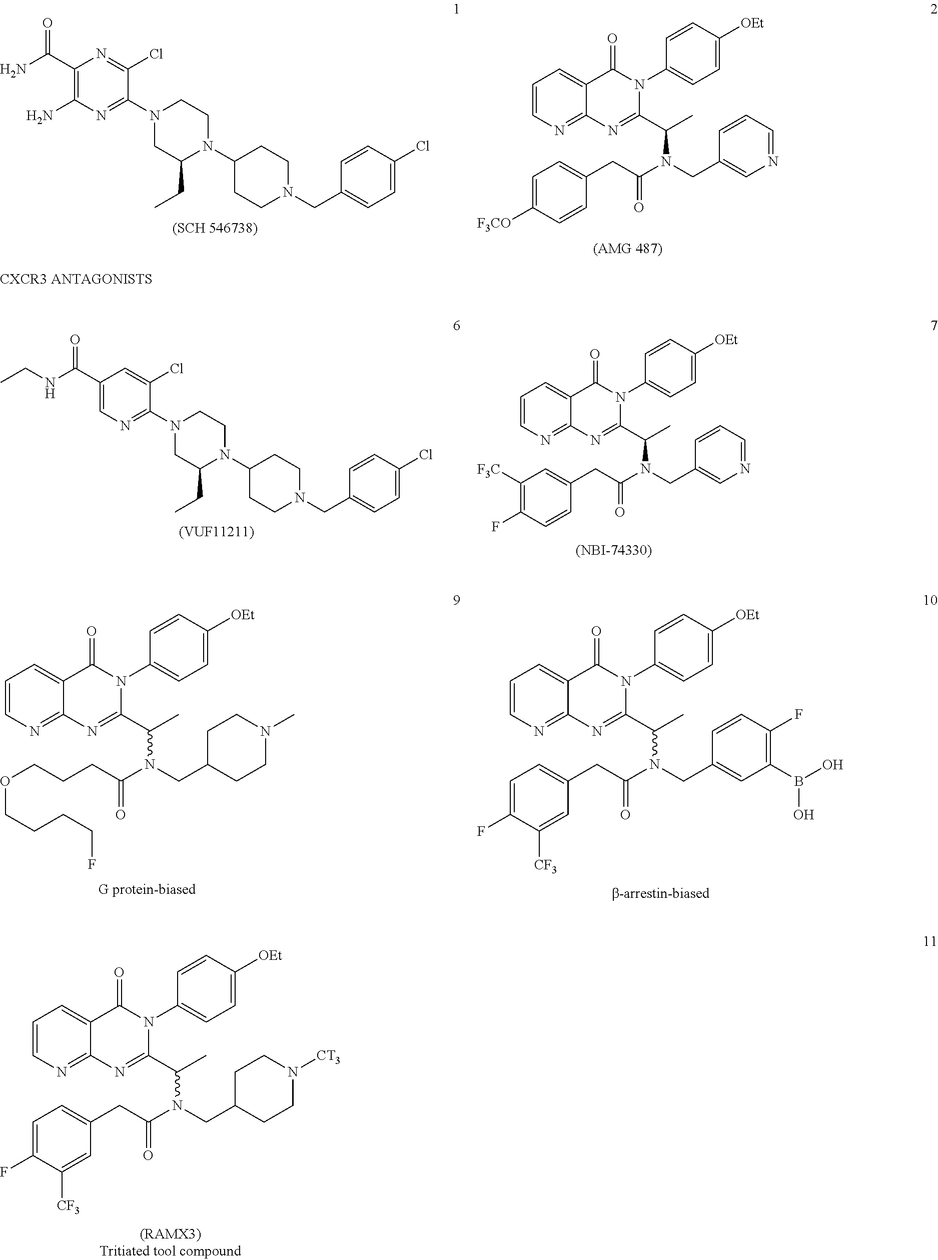

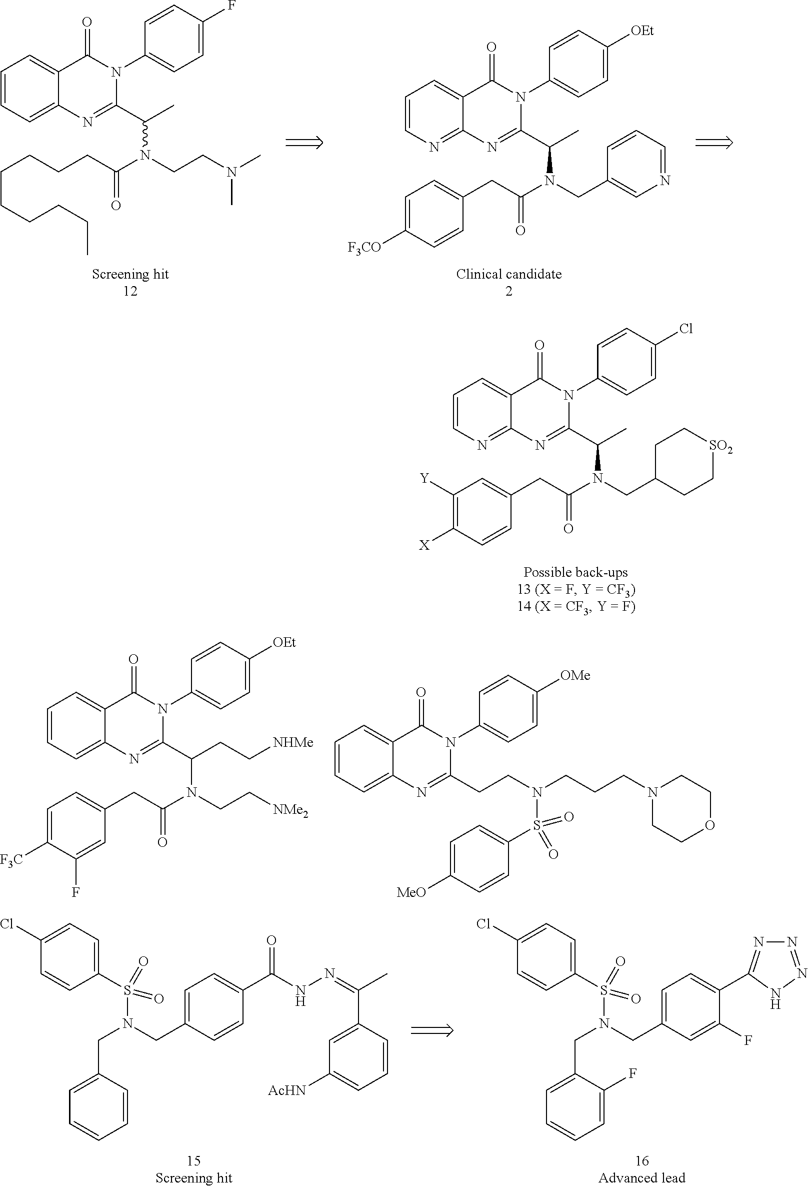

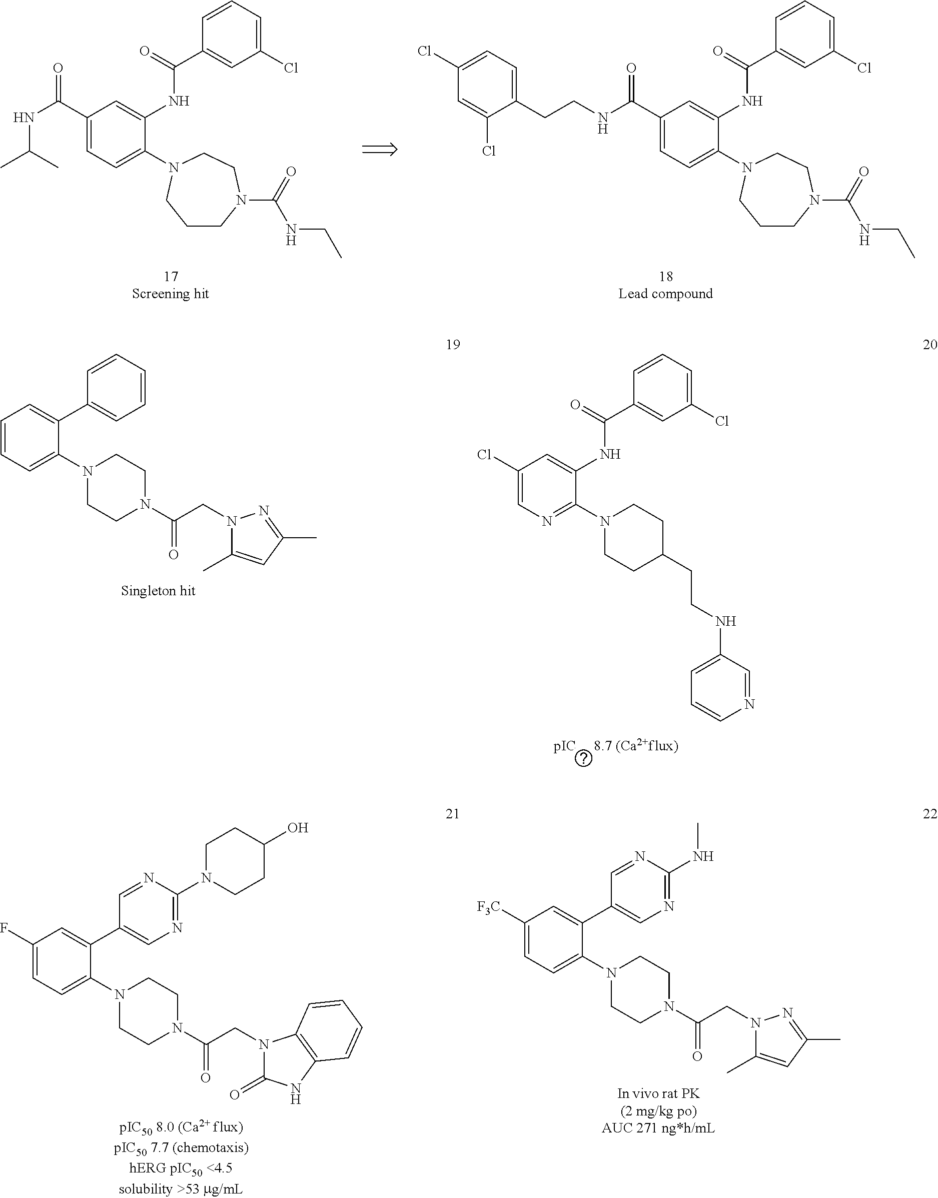 [SMILES: CCNC(=O)N1CCCN(c2ccc(C(=O)NC(C)C)cc2NC(=O)c2cccc(Cl)c2)CC1 ; CCNC(=O)N1CCCN(c2ccc(C(=O)NCCc3ccc(Cl)cc3Cl)cc2NC(=O)c2cccc(Cl)c2)CC1 ; Cc1cc(C)n(CC(=O)N2CCN(c3ccccc3-c3ccccc3)CC2)n1 ; O=C(Nc1cc(Cl)cnc1N1CCC(CCNc2cccnc2)CC1)c1cccc(Cl)c1 ; O=C(Cn1c(=O)[nH]c2ccccc21)N1CCN(c2ccc(F)cc2-c2cnc(N3CCC(O)CC3)nc2)CC1 ; CNc1ncc(-c2cc(C(F)(F)F)ccc2N2CCN(C(=O)Cn3nc(C)cc3C)CC2)cn1]

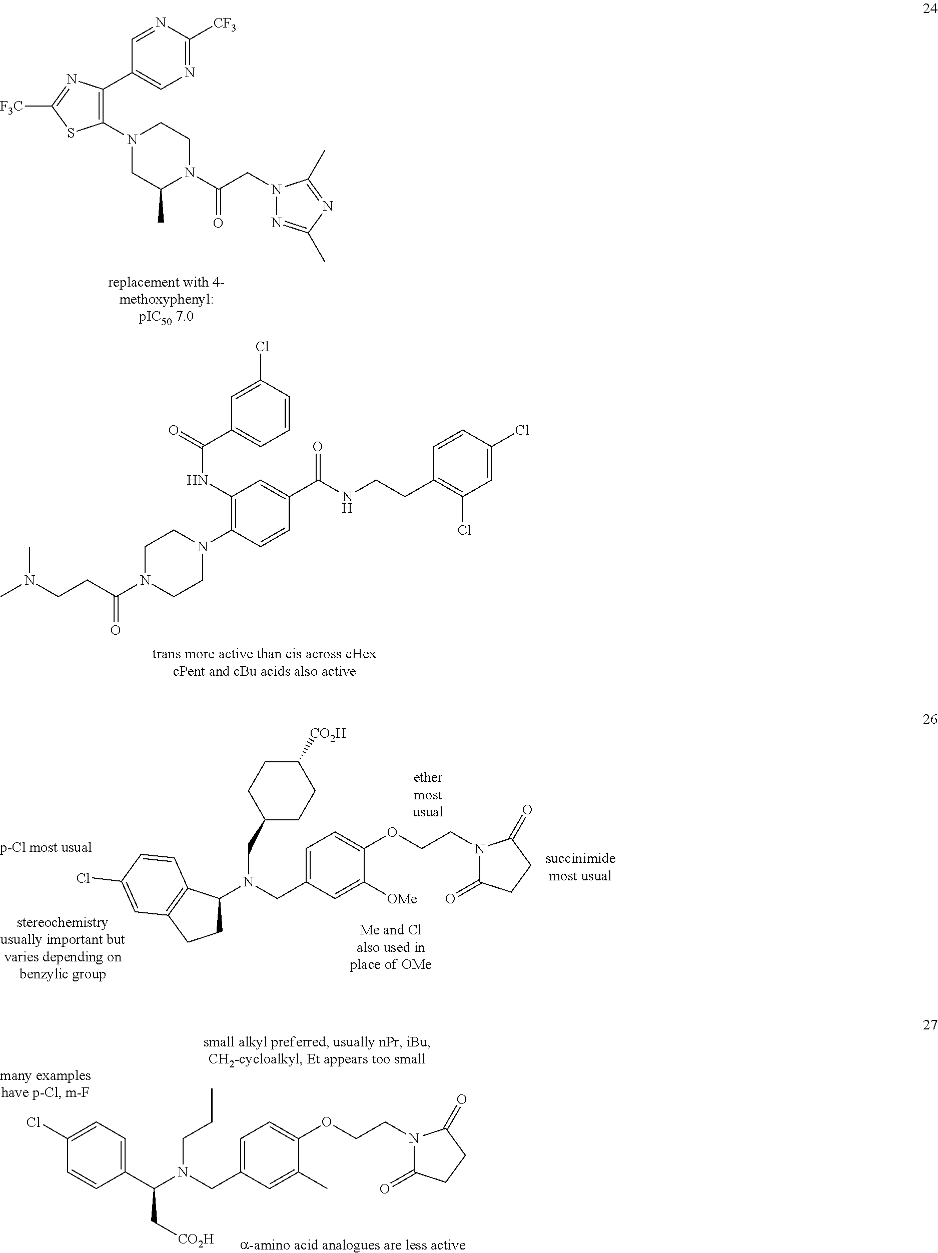 [SMILES: Cc1nc(C)n(CC(=O)N2CCN(c3sc(C(F)(F)F)nc3-c3cnc(C(F)(F)F)nc3)C[C@@H]2C)n1 ; CN(C)CCC(=O)N1CCN(c2ccc(C(=O)NCCc3ccc(Cl)cc3Cl)cc2NC(=O)c2cccc(Cl)c2)CC1 ; COc1cc(CN(C[C@H]2CC[C@H](C(=O)O)CC2)[C@H]2CCc3cc(Cl)ccc32)ccc1OCCN1C(=O)CCC1=O ; CCCN(Cc1ccc(OCCN2C(=O)CCC2=O)c(C)c1)[C@@H](CC(=O)O)c1ccc(Cl)cc1]

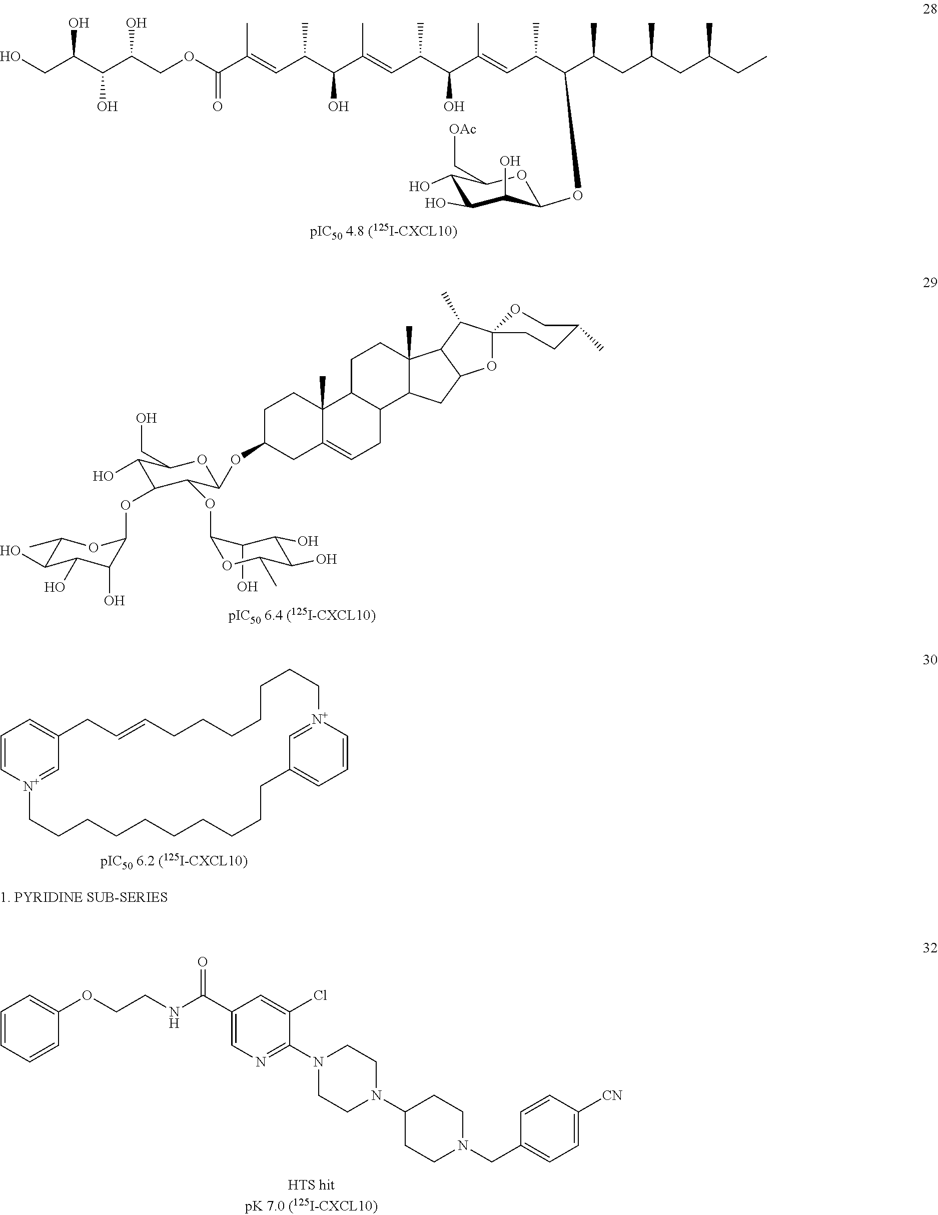

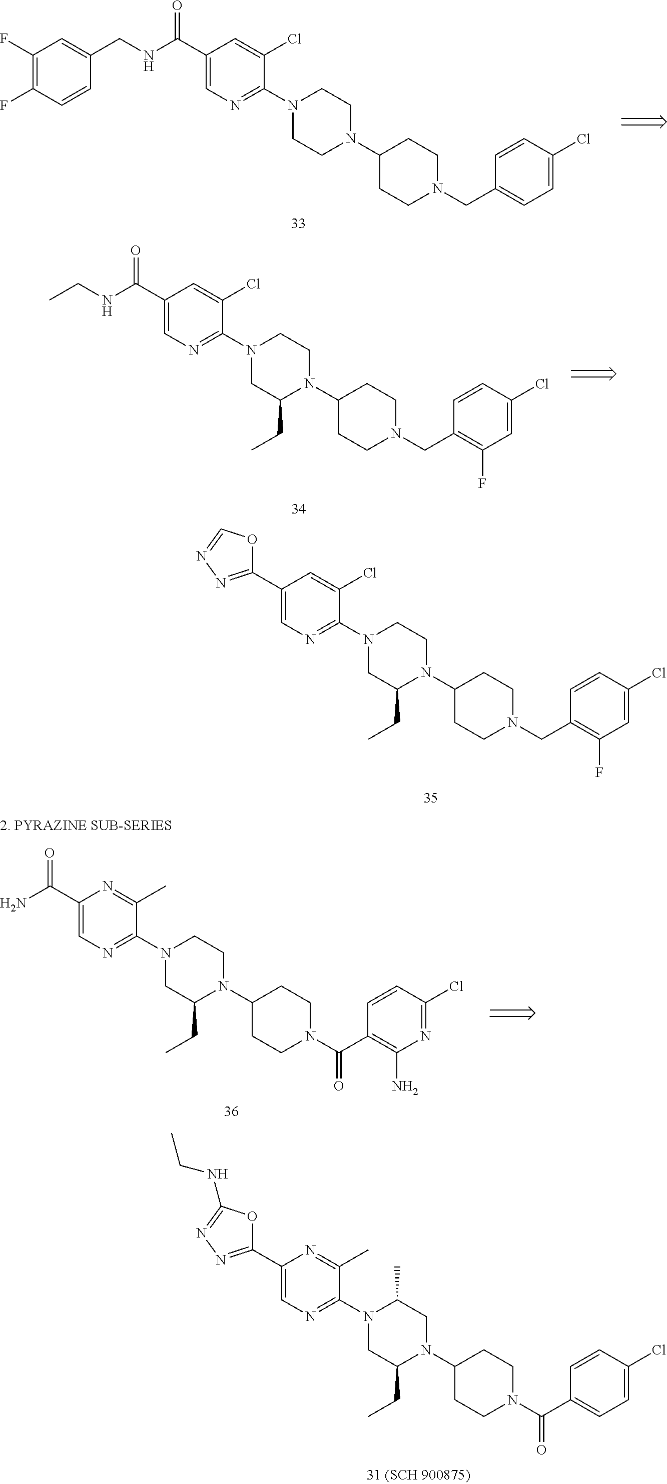 [SMILES: O=C(NCc1ccc(F)c(F)c1)c1cnc(N2CCN(C3CCN(Cc4ccc(Cl)cc4)CC3)CC2)c(Cl)c1 ; CCNC(=O)c1cnc(N2CCN(C3CCN(Cc4ccc(Cl)cc4F)CC3)[C@@H](CC)C2)c(Cl)c1 ; CC[C@H]1CN(c2ncc(-c3nnco3)cc2Cl)CCN1C1CCN(Cc2ccc(Cl)cc2F)CC1 ; CC[C@H]1CN(c2ncc(C(N)=O)nc2C)CCN1C1CCN(C(=O)c2ccc(Cl)nc2N)CC1 ; CCNc1nnc(-c2cnc(N3C[C@H](CC)N(C4CCN(C(=O)c5ccc(Cl)cc5)CC4)C[C@H]3C)c(C)n2)o1]

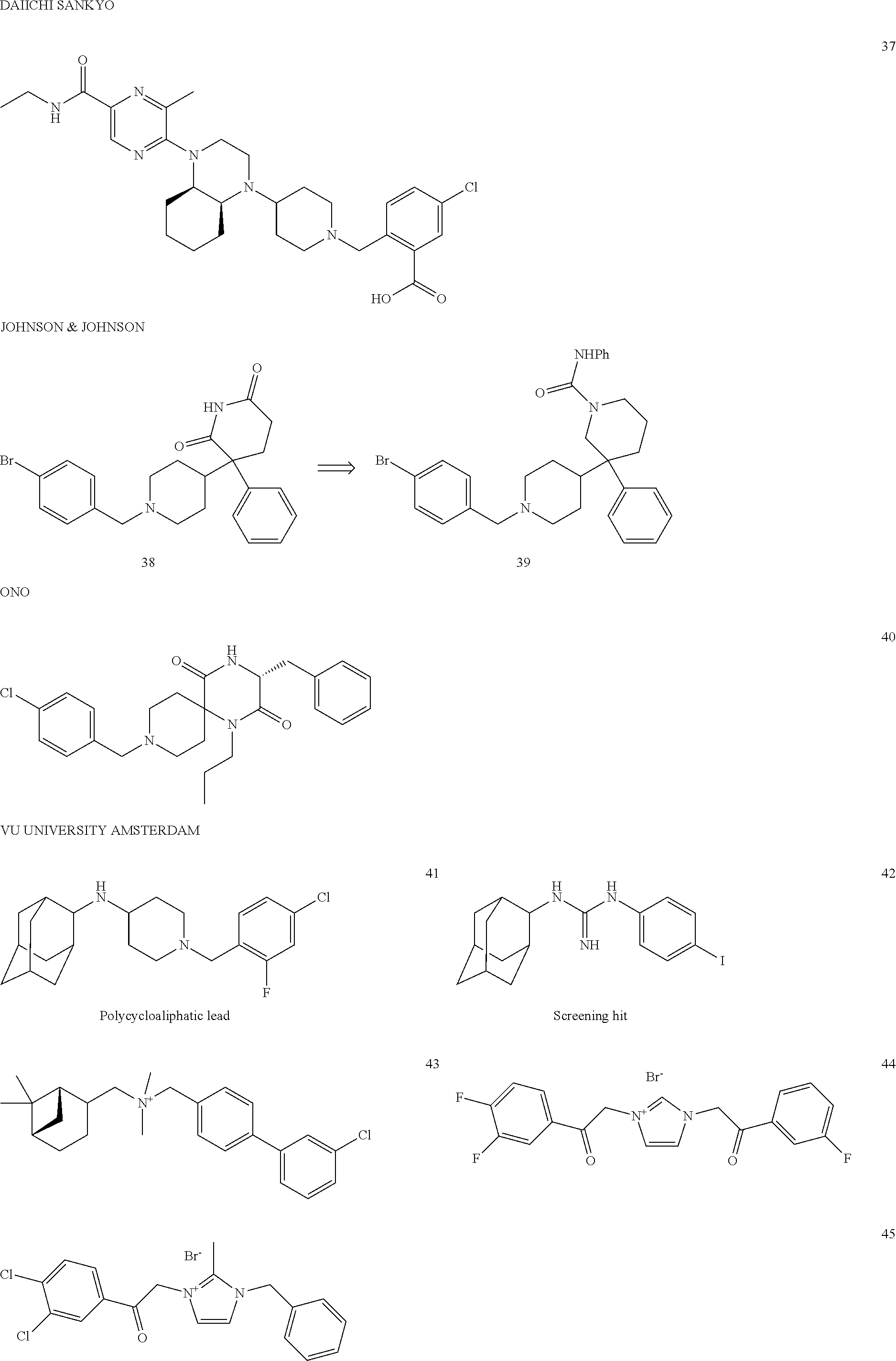

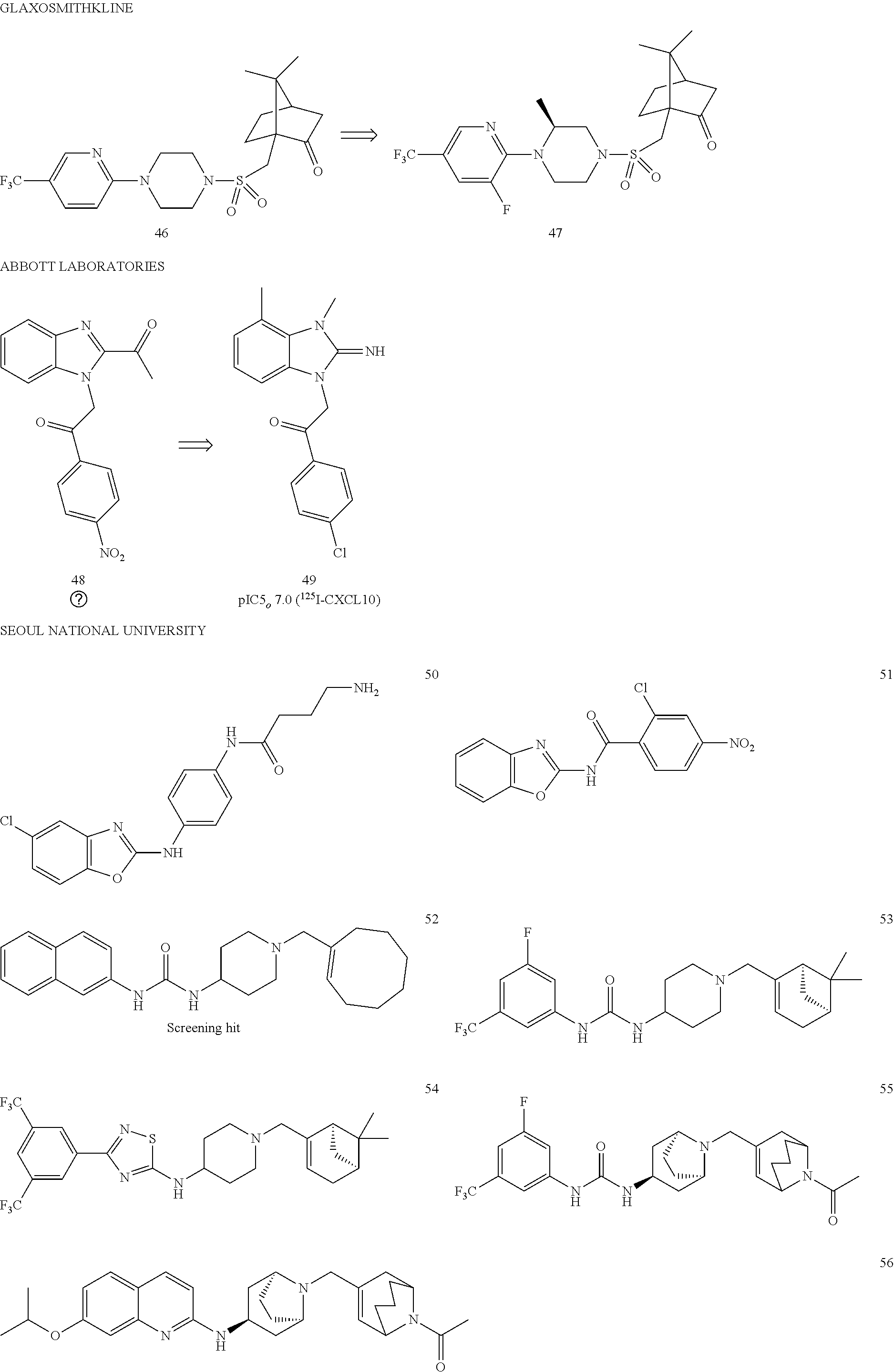

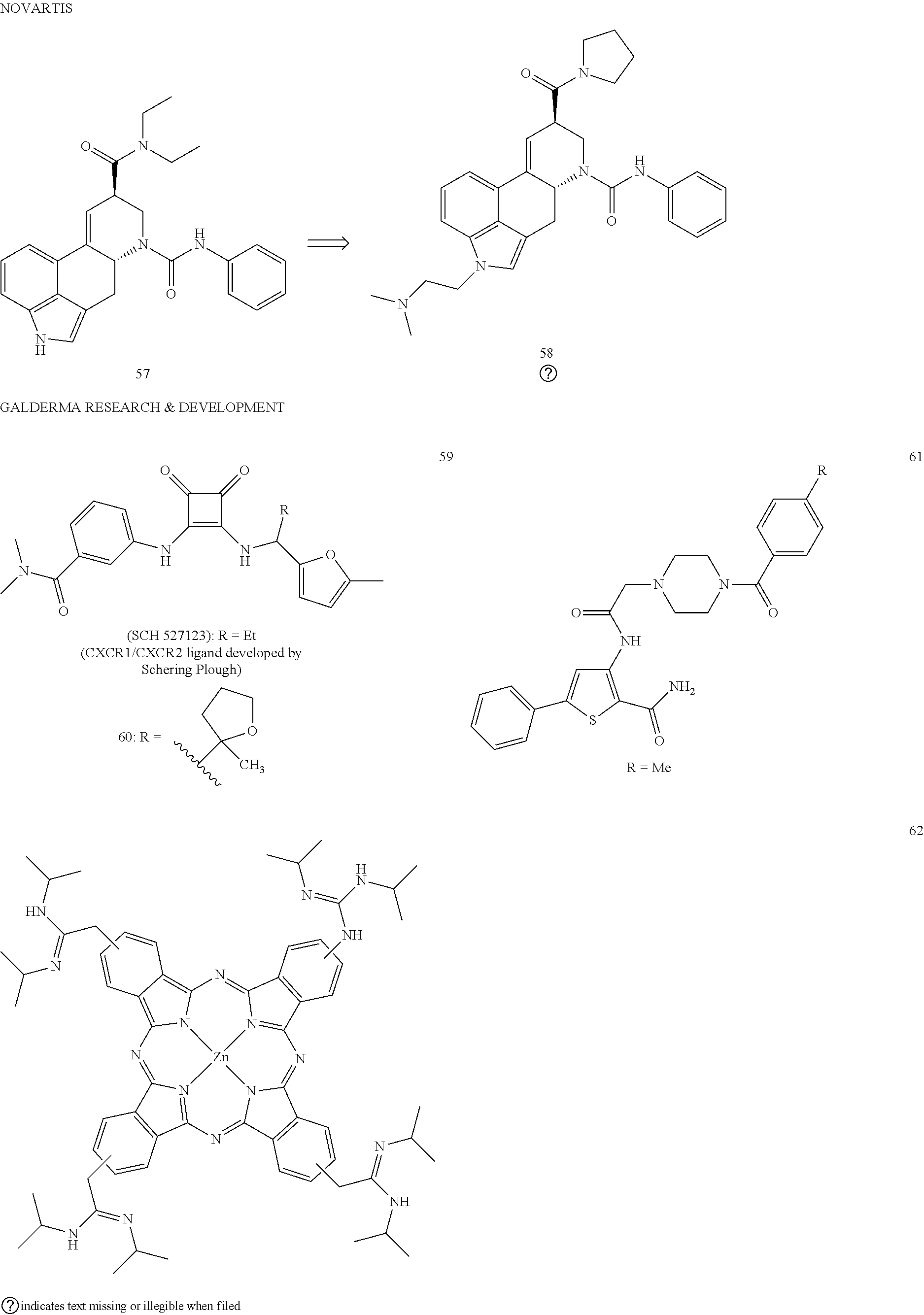

Other small molecule antagonists are found, for example, in US20060036093, WO2009/105435, which all are incorporated by reference in their entirety for all purposes.

Any of the foregoing antibodies or fragments thereof (collectively antibodies) can be engineered for use in humans by methods such as, for example, chimerization, humanization, humaneering, etc, which are known in the art.

In addition, any of the foregoing antibodies or fragments thereof (collectively antibodies) can include a protracting moiety that extends a half-life (T_{1/2}) or/and the duration of action of the antibody. The protracting moiety can extend the circulation T_{1/2}, blood T_{1/2}, plasma T_{1/2}, serum T_{1/2}, terminal T_{1/2}, biological T_{1/2}, elimination T_{1/2} or functional T_{1/2}, or any combination thereof, of the antibody. One or more protracting moieties can be combined (covalently or noncovalently) with an antibody. Protracting moieties include, for example, hydrophilic polymers (e.g., PEG, dextran, etc.), a synthetic polymer, glycosylation, human serum albumin (HSA) or a portion thereof (e.g., domain III) that binds to the neonatal Fc receptor (FcRn), or a carboxy-terminal peptide (CTP).

Additional agents (drugs, foods and other molecules) that can alter iAge by affecting genes involved in systemic chronic inflammation comprising MIG, TNFSF10, IFNg, CCL11 or CXCL1 are listed below in Tables 2, 3 and 4. These molecules were obtained using methods described below with a combined confidence score >500 (q value of <0.05, nominal p value of <0.005) Table 2 shows drugs and other molecules that can change iAge by interacting with immune genes involved in the inflammatory response including MIG, TNFSF10, IFNg, CCL11 or CXCL1 and changing the levels of these proteins in the subject. Table 3 shows food compounds and other molecules that can change iAge by interacting with immune genes involved in the inflammatory response including MIG, TNFSF10, IFNg, CCL11 or CXCL1 and changing the levels of these proteins in the subject. Table 4 shows drugs that can upregulate or downregulate MIG, TNF SF10, IFNg, CCL11 or CXCL1, and whether the up- or down-regulation is beneficial (lowers) or detrimental (raises) to iAge. Other drugs and other molecules that interact with genes/proteins involved in inflammation and/or the inflammatory response can be used to reduce the iAge of the subject through indirect effects on the levels of the iAge markers which are described above. Other food compounds or other molecules that interact with genes/proteins involved in inflammation and/or the inflammatory response can be used to reduce the iAge of the subject through indirect effects on the levels of the iAge markers which are described above.

Methods for Determining the Effect of a Substance on iAge

Compounds that can modify iAge are identified from DrugBank and FooDB using a compound-gene interaction database, machine learning for drug repurposing and food compound mapping for anti-inflammatory activity, and medication usage studies from the 1KIP cohort.

The STITCH database v. 5.0 can be used as the compound-gene interaction database to find immune genes with which a drug or food compound interacts. Compound-protein interactions are extracted from the STITCH database v5.0 by matching the InChI keys of drugs/food compounds. STITCH collects information from multiple sources and individual scores from each source are combined into an overall confidence score.

An immune gene set (n=4275) is obtained from the HIM CHip panel. The immune gene set is then matched with the two compound-gene interaction datasets above to extract immune genes that interact with drugs or food compounds. The immune gene set is used in the STITCH database, and FDA-approved drugs (n=1692) are selected from the DrugBank database and food compounds (n=7962) are selected from the FoodDB database as previously described in Veselkov et l., Sci Rep. 2019; 9: 9237, PMCID: PMC6610092, which is incorporated by reference in its entirety for all purposes.

All interactions with a combined confidence score of less than 500 are removed. The Ensemble peptide identifiers for protein are then converted to HGNC gene symbols using Biomart (version 2.42.0). As a result, there are 1617 immune genes interact with drugs and 1774 immune genes interact with food compounds. After pre-processing, two data sets are obtained: i) drug-gene interaction dataset containing 1670 drugs, 9642 genes with 118,342 interaction ii) food compound—gene interaction dataset containing 3447 compounds, 10,942 genes, and 166,431interactions.

To investigate compound—immunity association, statistical significance for the enrichment of compound genes in the immune gene set is calculated using Fisher's exact test. The universal gene set contains all genes that interact with at least one compound. The compound with low p-value interacts with a higher proportion of the immune gene set than that expected by chance. The statistical analysis is performed using R.

FDA-approved drugs (n=1692) are selected from the DrugBank database as previously described in Veselkov et l., Sci Rep. 2019; 9: 9237, PMCID: PMC6610092, which is incorporated by reference in its entirety for all purposes. The set of drugs is mapped to the DrugCentral database via InChI keys to identify drugs indicated for anti-inflammatory treatment (n=49), that are denoted as ‘positive class’. All drugs with no known association with anti-inflammatory activity are denoted as ‘negative class’. This set of drugs will be used as a training set to train machine learning models. Food compounds (n=7962) are selected from the FoodDB database as previously described. The set of food compounds will be used as a test data set.

Compound-protein interactions are extracted from the STITCH database v5.0 by matching the InChI keys of drugs/food compounds. STICH collects information from multiple sources and individual scores from each source are combined into an overall confidence score. The threshold for the significant score is not set at a fixed value and considered as an adjustable parameter for ML model optimization.

Gene-gene interactions are extracted from public sources including STRING, UniProt, COSMIC, BioPlex, and NCBI Gene as previously described in Veselkov et l., Sci Rep. 2019; 9: 9237, PMCID: PMC6610092, which is incorporated by reference in its entirety for all purposes. This results in a gene-gene interactome dataset containing 20,256 genes with ˜11 million interactions.

The gene profile for each compound is represented as a sparse matrix, in which a ‘1’ indicates genes that directly interact with the compound and a ‘0’ for all other genes. The network propagation (Random Walk with Restart) algorithm is then applied to spread this gene profile on to the human interactome. As a result, a genome-wide profile of gene scores is obtained for each compound. The restart parameter ‘c’ is considered as an adjustable parameter for ML model optimization.

ML can use Linear SVM as a classifier for optimization. The interaction score threshold can be set at 600, the restart parameter ‘c’ for network propagation can be set at 0.1. Linear SVM can be used to identify anti-inflammatory drugs based on their genome-wide profile obtained from network propagation. The regularization parameter ‘C; can be optimized during the model training using a nested cross-validation strategy. The F-score, that balances sensitivity and specificity, is used to evaluate the outcome of each model. The best model is defined as a model with the highest F-score.

The anti-inflammatory ‘likeness’ of drugs is calculated using the selected model. These values are used to identify potential drugs for anti-inflammatory repurposing. Similarly, the selected model is applied to the food compound dataset. The probability estimates for the anti-inflammatory activity of each food compound are calculated. Food compounds with high anti-inflammatory probability (i.e, >0.8) are selected for validation.

Medication prescribed for 1KIP cohorts are divided into 23 groups. Medication usage is represented as a sparse matrix, in which ‘1’ indicates the patient took at least one of the drugs in that group and ‘0’ indicates none of the drugs in that group was not taken.

Several analyses were performed as follows: build a model to predict iAge based on medication usage using lasso method implemented in ‘glmnet’ package; study correlation between medication usage and cytokine/chemokine levels using PLS method implemented in ‘pls’ package; and build a model to predict cytokine/chemokine levels based on medication usage using lasso in ‘glmnet’ and lasso with interaction in ‘glinternet’.

Additional ways to find agents that can lower iAge include a deep neural network approach implemented using DeepCOP (Woo et. al. 2019, Bioinformatics 36(3):813-818, which is incorporated by reference in its entirety for all purposes). Level 5 expression scores from the LINCS L1000 study can be used to label genes as up- or down-regulated if scores were more than a 50% perturbation above the top-threshold. Compounds in LINCS L1000 and compounds from FooDB were represented using calculated Morgan Fingerprints from SMILES using RDkit. Genes were represented as commonly occurring gene ontologies (at least three times). The deep neural network was trained on LINCS L1000 compounds on CD34 and HUVEC cells, independently, using the target genes plus the imputed expression for the genes that encode the Inflammatory Age proteins. Predicted expression probabilities for the compounds in FooDB were used to score these interventions. Compounds that only upregulate anti-inflammatory markers and downregulate pro-inflammatory markers are considered as possible interventions.

The network propagation algorithm can be implemented using the method in (Veselkov., et al, 2019, Scientific Reports (9) 9237). The assumption is that compounds with similar network profiles would have similar effect of regulation of a certain gene. Compound-gene interactions can be extracted from the STITCH database v5.0. STITCH collects information from multiple sources and individual scores from each source are combined into an overall confidence score. Gene-gene interactions can be extracted from public sources including STRING, UniProt, COSMIC, BioPlex, and NCBI Gene as previously described in Veselkov et al, 2019. This results in a gene-gene interactome dataset containing 20,256 genes with ˜11 million interactions.

The gene profile for each compound can be represented as a sparse matrix, in which a ‘1’ indicates genes that directly interact with the compound and a ‘0’ for all other genes. The network propagation (Random Walk with Restart) algorithm can then applied to spread this gene profile onto the gene-gene interactome. As a result, a genome-wide profile of gene scores can be obtained for each compound. The restart parameter ‘c’ can be considered as an adjustable parameter for ML model optimization.

The LINCS compounds that are already known the regulation direction for a certain gene can be used to train a linear SVM classification model using network propagation profile as features. The regularization parameter ‘C; can be optimized during the model training using a nested cross-validation strategy. The F-score, that balances sensitivity and specificity, can be used to evaluate the outcome of each model. The best model can be defined as a model with the highest F-score. The selected model can then be applied to the FooDB compound dataset. The probability estimates for the desired regulation direction of each food compound can be calculated and can be used as a score to determine the probability of each intervention.

Treating Immunotypes to Lower iAge

Table 2 below lists the protein markers that can have their levels changed to improve the iAge for patients in the different immunotypes.

TABLE 2
Protein Markers to Change for Each Immunotype

| Protein Levels to change | ImmunoType |
|---|---|
| TRAIL | SH1 |
| GroA/IFNG/MIG | SH2, N1 |
| None | SH3 |
| Eotaxin/MIG | SH4 |
| TRAIL/IFNG | N2 |
| Eotaxin/GroA/IFNG | N3 |
| GroA/IFNG/TRAIL | N4 |
| Eotaxin/GroA/MIG/IFNG | N5 |
| Eotaxin/MIG/TRAIL | N6 |

Table 3 below shows GRAS (generally regarded as safe) compounds that can be used to lower each of the five protein markers in the Immunotypes.

TABLE 3
GRAS Compounds for Each Protein Marker

| Protein Marker | GRAS Compounds |
|---|---|
| TRAIL | Iron bisglycinate/biotin/caffeine |
| GroA | Iron bisglycinate/manganese chloride/niacin |
| IFNg | manganese chloride/beta carotene/leutin/zinc |
| | sulfate |
| MIG | vitamin d2/niacin/guar gum |
| Eotaxin | L-methionine/indole-3-carbinol/picetannol |

The level of one or more of the five protein markers can be improved for iAge by providing a patient with one or more of the GRAS compounds listed in Table 3. Table 2 above lists the proteins markers to improve for each immunotype. Table 3 identifies GRAS compounds that can be used for each protein marker to improve iAge. For example, a patient in Immunotype SH1 can be administered one or more of iron, biotin and/or caffeine. Table 4 below shows the GRAS compounds that can be used to improve iAge for the ten immunotypes.

TABLE 4
GRAS Compounds for Improving iAge of Each Immunotype

| Immunotype | intervention 1 | intervention 2 | intervention 3 | intervention 4 |
|---|---|---|---|---|
| 1-1 | Iron bisglycinate/ | | | |
| | biotin/caffeine | | | |
| 1-2, 2-1 | Iron bisglycinate/ | manganese | vitamin | |
| | manganese | chloride/beta | d2/niacin/ | |
| | chloride/niacin | carotene/leutin/ | guar gum | |
| | | zinc sulfate | | |
| 1-3 | | | | |
| 1-4 | vitamin d2/niacin/ | | | |
| | guar gum | | | |
| 2-2 | iron bisglycinate/ | manganese | | |
| | biotin/caffeine | chloride/beta | | |
| | | carotene/leutin/ | | |
| | | zinc sulfate | | |
| 2-3 | iron bisglycinate/ | manganese | L-methionine/ | |
| | manganese | chloride/beta | indole-3-carbinol/ | |
| | chloride/niacin | carotene/leutin/ | picetannol | |
| | | zinc sulfate | | |
| 2-4 | Iron bisglycinate/ | Iron bisglycinate/ | manganese | |
| | biotin/caffeine | manganese | chloride/beta | |
| | | chloride/niacin | carotene/leutin/ | |
| | | | zinc sulfate | |
| 2-5 | Iron bisglycinate/ | manganese | vitamin | L-methionine/ |
| | manganese | chloride/beta | d2/niacin/ | indole-3-carbinol/ |
| | chloride/niacin | carotene/leutin/ | guar gum | picetannol |
| | | zinc sulfate | | |
| 2-6 | Iron bisglycinate/ | vitamin | L-methionine/ | |
| | biotin/caffeine | d2/niacin/ | indole-3-carbinol/ | |
| | | guar gum | picetannol | |

Table 4 shows other agents that can be administered to patients to improve the levels of Eotaxin, GroaA, IFNg, MIG, and/or TRAIL and lower the patient's iAge. The agents to be used can depend upon the patient's immunotype (see Table 2, 3 and 4). Combinations of agents can be made (e.g., see Table 4) for individual immunotypes using agents from Table 3.

Additional agents that can be used to improve the levels of Eotaxin, GroaA, IFNg, MIG, and/or TRAIL and lower the patient's iAge are in Table 5. The agents in Table 5 can be used as described above to improve the levels of Eotaxin, GroaA, IFNg, MIG, and/or TRAIL and lower the iAge of patients in certain immunotypes. In addition, combinations of one or more of these agents in Table 5 can be used to improve the levels of Eotaxin, GroA, IFNg, MIG, and/or TRAIL and lower the iAge of patients in certain immunotypes.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1

### iAge Correlates with Naïve CD8(+) T Cells and with the Ex Vivo Jak-STAT Signaling Responses to Stimulation

The frequency of circulating naïve CD8(+) T cells decline with high iAge (A) and iAge can predict poor ex vivo Jak-STAT signaling responses to stimulation (B and C). A total of 96 cytokine-cell-STAT combinations were analyzed with respect of a subject's iAge. These included eight cell types: B cells, CD4(+) T cells (and their CD45(+) and (−) subsets), CD8(+) T cells (and their CD45(+) and (−) subsets), and monocytes; four cytokines: Interleukin-6 (IL-6), IL-10, IL-21 and Interferon-alpha; and three STAT proteins (STAT1, 3 and 5). FIG. 1B: volcano plot, result of a multiple regression analysis with permutation tests to estimate false discovery rates (Benjamini-Hochberg FDR) (y-axis) as a function of the regression coefficients obtained for iAge after adjusting for Age, Gender and cytomegalovirus status. FIG. 1C: normalized ex vivo CD8(+) T cell phosphor-STAT-1 responses to Interleukin-6. The lower tertile for iAge shows significantly more robust responses than the higher tertile for iAge (C).

iAge is negatively correlated with naïve CD8(+) T cells and with the ex vivo Jak-STAT signaling responses to stimulation.

### Example 2

### Stratification of Cancer Patients Using iAge and CRS

A blood sample is obtained from patients prior to immunotherapy treatment. Serum and immune cells are separated by standard methods. Serum samples are used to measure protein concentration for inflammatory age (iAge) determination; and cells are cytokine-stimulated ex vivo to measure phosphorylation of intracellular signal transducer and activator of transcription (STAT) proteins to derive a cytokine response score (CRS). iAge and CRS can independently predict patient's response to immunotherapy treatment. FIG. 2 shows a flow diagram of this process.

iAge and CRS can be used to stratify cancer patients prior to treatment as responders versus non-responder for immunotherapy.

### Example 3

### Stratification of Cancer Patients Using iAge

iAge can be used to classify cancer patients into responder and non-responders to immunotherapy treatment (A), and to derive iAge individual inflammatory protein signature (barcode), which is fed to iAge personalized recommendation engine to create an individualized initial therapy aimed to reduce iAge, inform medical decision and hence, convert those non-responder patients into responder patients (suitable for immunotherapy) (B). FIG. 3 shows a flow diagram of this process.

iAge is used to stratify patients for cancer immunotherapy and help convert non-responders into responder for immunotherapy.

### Example 4

### Endothelial Cells Derived from hiPSCs Produce MIG

Human induced pluripotent stem cells (hiPSCs) were obtained from isolated fibroblasts (N=5, in duplicates) using the Yamanaka factors (Takahashi and Yamanaka, 2006) and differentiated them into endothelial cells (hiPSC-ECs) under well-defined conditions as previously described (Hu et al., 2016).

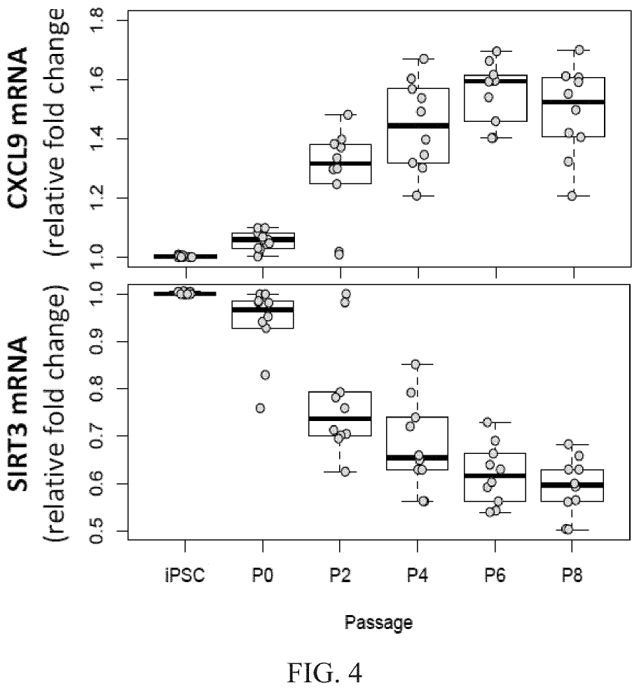

Expression levels of MIG and SIRT3 were measured by RT-PCR. A significant age-dependent increase in MIG mRNA expression levels is observed (P<0.01), which reaches a plateau after the sixth cell passage. (See FIG. 4) Concomitant with the increase in MIG, down-regulation in SIRT3 mRNA can be observed after the second cell passage (P<0.01). (See FIG. 4)

### Example 5

### Expression of CXCR3 in Endothelial Cells

Human induced pluripotent stem cells were made as described in Example 4. Expression of the MIG receptor, CXCR3, was measured in young cardiomyocytes derived from hiPSCs (hiPSC-CM) as well as in hiPSC-ECs (endothelial cells derived from hiPSC), HUVEC cells, freshly isolated fibroblasts and hiPSCs.

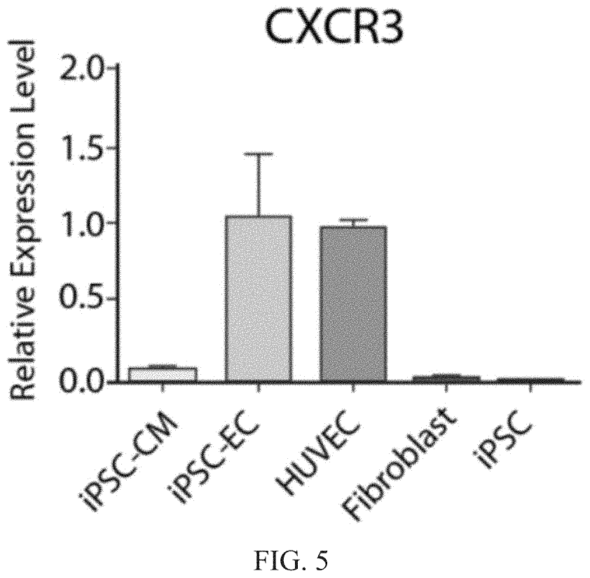

Elevated expression of CXCR3 is observed in hiPSC-ECs, HUVEC cells but not in other cell types (F) suggesting that the endothelium but not other cell subsets is a target of MIG and potentially other CXCR3 ligands as well. (See FIG. 5)

### Example 6

### MIG Impairs Endothelial Cell Function

Mouse thoracic aortas were carefully dissected, and vessels were cut into small rings and mounted on an isometric wire myograph chambers (Danish Myo Technology) and subjected to a normalization protocol. Following normalization, the vessels were incubated with either PBS or different concentrations of recombinant mouse MIG (R&D systems, catalog number 492-MM). A concentration-dependent contraction curve was created by accumulative application of the prostaglandin agonist U46619. Subsequently, concentration-dependent relaxation curves of Acetylcholine were conducted on these vessels and percent relaxation calculated for each dose.

FIG. 6 shows a line graph of percent relaxation of mouse thoracic aortic sections to Acetylcholine after exposure to different amounts of MIG. FIG. 6 shows impaired vascular reactivity with increasing concentrations of MIG. MIG causes a dose-dependent effect on vasorelaxation in treated aortas demonstrating that MIG impairs vascular function, and can contribute to arterial stiffness and premature aging of the cardiovascular system.

### Example 7

### Compositions for Treating Immunotypes

Tables 2 to 4 show agents that can be used to lower iAge by improving the levels of the markers Eotaxin, GroA, IFNg, MIG, and/or TRAIL. Table 3 shows how the treatment of certain markers correlates to immunotype, and Table 4 shows combinations of agents that can administer to patients in the different immunotypes to improve their iAge.

Table 6 below, shows dosages that can be used in formulating the agents into compositions that can be administered to patients of certain immunotypes.

TABLE 6
Dose of Agents for Improving Levels
of an iAge Marker in a Patient

| Agent | Marker | Dosage per day | |
|---|---|---|---|
| Iron bisglycinate | TRAIL/GroA | 68 | mg |
| manganese chloride | IFNG/GroA | 9 | mg |
| vitamin D2 | MIG | .05 | mg |
| niacin | MIG/GroA | 150 | mg |
| guar gum | MIG | 1000 | mg |
| indole-3-carbinol | Eotaxin | 100 | mg |
| L-methonine | Eotaxin | 250 | mg |
| Piceatannol | Eotaxin | 5 | mg |
| biotin | TRAIL | 5 | mg |
| caffeine | TRAIL | 100 | mg |
| beta-carotene | IFNG | 15 | mg |
| leutin | IFNG | 20 | mg |
| zinc sulfate | IFNG | 220 | mg |

For example, treatment of patients from Immunotypes SH2 or N1 can improve the levels of GroA, IFNg, and MIG. A composition for improving GroA can have iron bisglycinate, manganese chloride, and niacin. The dose of each of these per day is 245 mg iron bisglycinate, 9 mg manganese chloride, and 250 mg niacin. A composition for improving IFNg can have manganese chloride, beta carotene, leutin, and zinc sulfate. The dose of each of these is 15 mg beta-carotene, 20 mg leutin, 220 mg zinc sulfate, and the manganese chloride doses is the same as for GroA. A composition for improving MIG can have vitamin d2, niacin, and guar gum. The dose of each of these is 0.05 mg vitamin d2, 1000 mg guar gum, and dose of niacin is the same as for GroA. These compositions can be combined into one dosage form, placed in separate dosage forms, or the components of each can be mixed and matched into separate dosage forms for administering to a patient.

[symbol]

Using Tables 2, 3, and 4 combined with the dosing in Table 5 one can make dosage forms containing agents that can improve the iAge of patients in any of the immunotypes as was shown above for Immunotypes SH2 or N1.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for modifying an iAge of a subject, comprising the steps of: identifying the immunotype of the subject; and administering an effective amount of an agent that changes an amount of an iAge marker for the subject, whereby the agent lowers the iAge for the subject.

2. The method of claim 1, wherein the molecule is a drug.

3. The method of claim 1, wherein the agent changes the level of an Eotaxin, a GroA, an IFNg, a MIG, or a TRAIL.

4. The method of claim 3, wherein the agent downregulates the MIG whereby the iAge of the subject is reduced.

5. The method of claim 4, wherein the agent is a vitamin d2, a niacin, or a guar gum.

6. The method of claim 1, further comprising including the agent in a composition for changing the amount of the iAge marker.

7. The method of claim 6, wherein the composition downregulates the MIG and wherein the composition comprises a vitamin d2, a niacin, and a guar gum.

8. The method of claim 3, wherein the drug upregulates the IFNg whereby the iAge of the subject is decreased.

9. The method of claim 8, wherein the agent is a manganese chloride, a beta carotene, a leutin, or a zinc sulfate.

10. The method of claim 6, wherein the composition upregulates the IFNg and wherein the composition comprises a manganese chloride, a beta carotene, a leutin, and a zinc sulfate.

11. The method of claim 3, wherein the agent downregulates the Eotaxin whereby the iAge of the subject is decreased.

12. The method of claim 11, wherein the agent is an L-methionine, an indole-3-carbinol, or a picetannol.

13. The method of claim 6, wherein the composition downregulates the Eotaxin and wherein the composition comprises an L-methionine, an indole-3-carbinol, and a picetannol.

14. The method of claim 3, wherein the drug upregulates the GroA whereby the iAge of the subject is decreased.

15. The method of claim 14, wherein the agent is an iron bisglycinate, a manganese chloride, or a niacin.

16. The method of claim 6, wherein the composition upregulates the GroA and wherein the composition comprises an iron bisglycinate, a manganese chloride, and a niacin.

17. The method of claim 3, wherein the agent upregulates the TRAIL whereby the iAge of the subject is decreased.

18. The method of claim 14, wherein the agent is an iron, a biotin, or a caffeine.

19. The method of claim 6, wherein the composition upregulates the TRAIL and wherein the composition comprises an iron bisglycinate, a biotin, and a caffeine.

20. The method of claim 20, wherein the immunotype is a SH1, a SH2, a SH4, a N1, a N2, a N3, A N4, a N5, or a N6.
